(19) United States
(12) Patent Application Publication
(10) Pub. No.: US 2011/0060222 A1
(43) Pub. Date: Mar. 10, 2011

(54) METHOD AND APPARATUS FOR CHARACTERIZING INHOMGENEITIES USING AXIAL SHEAR STRAIN ELASTOGRAPHY

(75) Inventors: Arun K. THITTAI, Houston, TX (US); Belfor GALAZ, Santiago (CL); Jonathan OPHIR, Austin, TX (US)

(73) Assignee: BOARD OF REGENTS OF THE UNIVERSITY OF TEXAS SYSTEM, Austin, TX (US)

(21) Appl. No.: 12/879,385

(22) Filed: Sep. 10, 2010

Related U.S. Application Data
(60) Provisional application No. 61/241,309, filed on Sep. 10, 2009.

(51) Int. Cl. A61B 8/00

(52) U.S. Cl. 600/438

(57) ABSTRACT

The present invention relates to systems and methods for characterizing inhomogeneities in deformable target bodies. More particularly, the disclosure relates to methods and systems for axial-shear strain elastography (ASSE) and their use in analyzing inclusions such as but not limited to, tumors as well as directing cancer therapies such as HIFU.

### CROSS-REFERENCE TO RELATED APPLICATIONS

This application claims the benefit under 35 U.S.C. §119(e) of U.S. Provisional Patent Application No. 61/241,309 filed Sep. 10, 2009 and entitled “Method Of Shear Strain Elastography Measurement And Imaging”, the disclosure of which is hereby incorporated herein by reference.

### STATEMENT REGARDING FEDERALLY SPONSORED RESEARCH OR DEVELOPMENT

This invention was made with U.S. Government support under Grant No. CA135580 awarded by the National Institutes of Health. The government has certain rights in the invention.

### BACKGROUND

1. Technical Field

The present invention relates to systems and methods for characterizing inhomogeneities in deformable target bodies. More particularly, the present invention relates to methods and systems for axial-shear strain elastography (ASSE) and their use in analyzing inclusions such as but not limited to, tumors as well as directing medical therapies.

2. Background

Ultrasound (US) elastography (see for example, Ophir J, Cèspedes I, Ponnekanti H, Yazdi Y, and Li X. Elastography: a method for imaging the elasticity of biological tissues. Ultrasonic Imaging; 13(2):11-13: 1991 and U.S. Pat. Nos. 5,107,837; 5,178,147; 5,293,870; and 5,474,070) is now a well-established technique. Elastography has been developed as a technology to visualize stiffness variations within a soft tissue region with the premise that if certain tissue regions have different stiffness parameters than others, the level of strain in those regions will generally be higher or lower than those in the surrounding tissue; a stiffer tissue region will generally experience less axial strain than a softer one.

Elastography involves acquiring US (RF and/or envelope) signals from an imaging plane in a target body before and after a small quasi-static compression is applied from within or without the target body and computing all the local axial displacements in the imaging plane. The gradients of these displacements are then used to generate a map of the local tissue axial strains and this strain map is referred to as an axial elastogram (Ophir J, Alam S K, Garra B S, Kallel F, Konofagou E E, Krouskop T A, and Varghese T. Elastography: Ultrasonic Estimation and Imaging of Elastic Properties of Tissues. Proc. Instn. Mech. Engrs. (UK) 1999; 213 (H3): 203-233).

Most commonly, the displacement profile along the direction of compression in a target body is accurately estimated due to the high sampling rate possible in that direction and the ability to use the ultrasound transducer as a tissue compression device. The gradient of the displacement is then used to generate a map of local tissue axial strains and this strain map is referred to as an axial elastogram. When the elastogram depicts axial strain values, it is referred to as an axial strain elastogram (ASE: see, for example, U.S. Pat. Nos. 6,270,459 and 7,779,692). The feasibility of using ASE to detect High Intensity Focused Ultrasound (HIFU) lesions has been demonstrated.

While it has been recognized that the application of lateral strain or elevational strain (both perpendicular to the axial strain) may be of value both in deducing qualities like Poisson's Ratio and in countering the effects of lateral motion in de-correlating the axial displacement of the tissue and shear strain images can also be obtained. A number of different methods have been used to obtain lateral strain elastography. Some of these techniques assume knowledge about the compressibility of the tissue (for example, Poisson's ratio), and thus cannot be used for measurements in which such tissue properties are to be determined. U.S. Pat. No. 6,270,459 describes a technique which interpolates between successive axial rays or echo signals to provide a basis for horizontal displacement measurement using a correlation technique.

U.S. Pat. No. 7,331,926, entitled “Ultrasonic Elastography Providing Axial, Orthogonal, And Shear Strain” describes a method of obtaining ultrasonic signals at a range of angles to derive both axial and lateral strain. This method exploits the ability to steer the US beam at several angles and thereby improve the quality of the displacement tracking estimates.

U.S. Pat. No. 7,601,122, entitled “Ultrasonic Elastography With Angular Compounding”, provides a method of obtaining both axial and lateral strain using multiple angles of ultrasonic measurement and an angle-dependent weighting factor. The compounding of the measurements from multiple angles improves the accuracy of the strain determinations.

U.S. Pat. No. 7,632,230, entitled “High Resolution Elastography Using Two Step Strain Estimation”, describes a multiple-step process in which successively finer samplings of data and smaller areas of data are evaluated to provide increasingly accurate displacement measurements, wherein each displacement measurement guides the determination of corresponding regions of comparison used in the next displacement evaluation.

However, it is reasonable to assume that any additional independent mechanical tissue parameters that can be obtained through elastography may either improve current elastographic performance and/or find utility in newer applications. Therefore, there is continuing need and interest in developing new methods and systems for elastography.

Presently described are methods of generating and analyzing ASSE such that a readily interpreted signal is used to reliably and noninvasively identify the boundaries of an inhomogenity and further characterize the nature of the bonding between the inhomogenity (for example, inclusion or lesion) and the surrounding environment, i.e. is it loosely or firmly bound. Furthermore in those instances where the inhomogeneity is a tumor these methods allow on to accurately and noninvasively obtain information regarding the malignant or benign nature of the tumor.

The disclosed methods also provide improved methods of reliably visualizing and identifying therapeutically induced tissue lesions, such as those created by HIFU treatment. A great benefit of the presently disclosed methods is the ease with which this information is obtained, presented and unambiguously interpreted.

### SUMMARY

The presently disclosed methods can be used to reliably and noninvasively determine the nature of the binding of an inhomogenity (for example, inclusion or lesion) and the surrounding environment (for example, tissue). High levels of maximum fill-in is used to identify loose binding between the inhomogenity and the surrounding environment, while low levels of maximum fill-in identify firm binding between the inhomogenity and the surrounding environment. Malignant tumors tend to be invasive and therefore firmly bound to the surrounding tissue. Benign tumors tend not to be invasive and are therefore loosely bound to the surrounding issues. Thus, the presently disclosed methods can be used to reliably and noninvasively determine the nature of a patient's tumor. These and other embodiments and potential advantages will be apparent in the following detailed description and drawings.

### BRIEF DESCRIPTION OF THE DRAWINGS

For a more detailed description of the preferred embodiment of the present invention, reference will now be made to the accompanying drawings, wherein:

FIG. 1 illustrates a schematic view of an exemplary imaging system suitable for practicing an embodiment of this disclosure.

FIG. 2 illustrates a schematic description of the set-up using a cross-section view showing different relative orientations between the transducer and inhomogeneity coordinate systems: Pitch angle (θ), Yaw angle (ø) and roll angle (Ψ).

FIG. 3 illustrates a schematic representation of the compression of a target body.

FIG. 4 illustrates a typical example of axial displacement map.

FIG. 5 illustrates typical axial strain elastogram obtained by computing the axial gradient of the axial displacement map of FIG. 4.

FIG. 6 illustrates a typical axial-shear strain elastogram and B-mode sonogram of, a pathologically-confirmed, benign tumor.

FIG. 7 illustrates an axial-shear strain elastogram on the left and a sonogram on the right of a benign tumor that is at a non-normal orientation with regards to the axis of compression.

FIG. 8a illustrates the steps for generating an axial-shear strain elastogram (ASSE) according to an embodiment of this disclosure.

FIG. 8b illustrates the application of ASSE according to an embodiment of this disclosure.

FIG. 9 is a sketch of the 2D geometrical parameters that describe the elliptical shaped inclusion used in this study, long-axis radius (a), short-axis radius (b), axial axis y (or axis of compression), lateral axis x and angle of orientation (θ) according to an embodiment of the present disclosure.

FIG. 10 shows an example set of images from malignant (top row) and benign (bottom row) in vivo cases used to define the Finite Element Modeling (FEM) geometry. The lesion margins that were manually traced (magenta) on the sonograms (a, e) were used to define the FEM geometry, which predicted the ideal axial-shear strain image (b, f). The ASSE obtained from the in vivo data is also shown (c, g). The segmented color-overlays of ASSE (c, g) on the corresponding sonograms (a, e) is also shown (d, h).

FIG. 11 shows ASSEs for firmly-bonded (top row) and loosely-bonded (bottom row) inclusions of symmetric circular shape (a, d), symmetric elliptical shape at θ=0° orientation (b, e), and asymmetric elliptical shape at θ=5° orientation (c, f).

FIG. 12 shows the (a) Effect of the orientation of a loosely-bonded (*) and firmly-bonded (∘) elliptical inclusion on the percent fill-in value. (b) Shows the plot of percent fill-in value only for the loosely-bonded inclusion case shown in (a) at a restricted x-axis (angle) range. Also, percent fill-in is shown at two additional (a/b=1 and 1.01) aspect ratios of the loosely-bonded inclusion.

FIG. 13 shows the: (a) effect of the inclusion aspect ratio (a/b) of a loosely (*) and firmly bonded (∘) elliptical inclusion on the percent fill-in value; and (b) the plot of percent fill-in value only for the loosely-bonded inclusion case shown in (a) at a restricted x-axis (a/b) range. Also, percent fill-in is shown at two additional (θ=0° and)5° orientations of the loosely-bonded inclusion.

FIG. 14 is a sketch of the geometrical parameters that describe the cross-sectional inclusion shape in the phantom: long-axis radius (a), short-axis radius (b) and angle of orientation (θ).

FIG. 15 is a schematic that illustrates alteration of (a) phantom 4 to obtain (b) phantom 5 with an elliptical inclusion orientation (θ) of 0°.

FIG. 16 is a flow chart description of the displacement tracking algorithm used in processing experimental and in vivo data in accordance with an embodiment.

FIG. 17 shows the sonogram and ASSE pair from (a) loosely-bonded and (b) firmly-bonded elliptical inclusion oriented at θ=0°.

FIG. 18 shows the sonogram and ASSE pair from (a) loosely-bonded and (b) firmly-bonded elliptical inclusion oriented at θ˜′−45° and 45°, respectively. Note the axial shear strain fill-in and contrasting inclusion boundary in (a).

FIG. 19 shows the sonogram and ASSE pair from the loosely-bonded elliptical inclusion phantom.

FIG. 20 shows the sonogram and ASSE pair from pathologically-confirmed (a) benign fibroadenoma (b) malignant tumor (DCIS) cases that appeared to have an elliptical inclusion oriented non-normally with axis of compression.

FIG. 21 shows the sonogram and ASSE pair from pathologically-confirmed benign fibroadenoma case that appeared to have an elliptical inclusion oriented non-normally with axis of compression. Observe the presence of axial-shear strain inside the lesion.

FIG. 22 shows the sonogram and ASSE pair from pathologically-confirmed (a) intraductal papilloma and (b) complex cyst that appeared to have an elliptical inclusion oriented non-normally with axis of compression. Observe the presence of axial-shear strain inside the lesion.

FIG. 23 is an illustrative flow diagram that demonstrates the iso-intensity contour segmentation method used in the HIFU-induced lesion area estimation from the ASE (left column, gray scale) and ASSE. The automatic lesion boundary determined at an iso-intensity threshold value of −3 dB is shown in the on bottom row. The manual lesion outline that was chosen as the starting position is shown in the images on top row.

FIG. 24 shows the sonogram (a), ASE (b) and ASSE (c) of a single HIFU-induced lesion case. The lesion boundary corresponding to two iso-intensity contours of −2 dB (solid line) and −6 dB (dashed line) are shown on the elastograms. Image size shown corresponds to 25 mm×20 mm.

FIG. 25 is a plot of the estimated HIFU-induced lesion area from ASE (∘) and ASSE (*) for the case shown in FIG. 23.

FIG. 26 shows the example of a case with two HIFU-lesion that appear to be well-separated on the different elastograms (a) ASE, (b) ASSE and (c) color-overlay of ASSE on ASE. Observe that the characteristic axial-shear strain pattern associated with each lesion (e.g. see ASSE in FIG. 2) is clearly identified in (c).

FIG. 27 shows the example of a case with two HIFU-lesion on elastograms (a) ASE, (b) ASSE and (c) color-overlay of ASSE on ASE. Observe the presence of a high-contrast axial-shear strain zone corresponding to the thin untreated region in the ASSE.

FIG. 28 shows the FEM-predicted axial-shear strain images of the 4 different two-inclusion models. The two 10 mm inclusions were located at the same depth but were laterally separated by (a) 10 mm, (b) 0.1 mm and (c) 50% overlap. In the forth model (d), the centers of the inclusions were offset vertically by 2.5 mm and were laterally separated by 0.1 mm.

### NOTATION AND NOMENCLATURE

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

As used herein, and unless otherwise indicated, the terms “treat”, “treating”, and “treatment” contemplate an action that occurs while a patient is suffering from a disease or disorder that reduces the severity of one or more symptoms or effects of the disease or disorder, or a related disease or disorder. Where the context allows, the terms “treat”, “treating”, and “treatment” also refers to actions taken toward ensuring that individuals at increased risk of a disease or disorder are able to receive appropriate surgical and/or other medical intervention prior to onset of disease or disorder. As used herein, and unless otherwise indicated, the terms “prevent”, “preventing”, and “prevention” contemplate an action that occurs before a patient begins to suffer from a disease or disorder that delays the onset of, and/or inhibits or reduces the severity of, a disease or disorder. As used herein, and unless otherwise indicated, the terms “manage”, “managing”, and “management” encompass preventing, delaying, or reducing the severity of a recurrence of a disease or disorder in a patient who has already suffered from such a disease or condition. The terms encompass modulating the threshold, development, and/or duration of the lung injury and disorders or changing how a patient responds to the disease or disorder.

As used herein, the term “ASSE” refers to axial-shear strain elastogram or axial-shear strain elastography. As used herein, the term “ASE” refers to axial strain elastogram or axial strain elastography. As used herein, the term “the orientation of the inclusion with respect to the axis of compression” would simply be referred as “inclusion orientation” or “orientation”, which is depicted by the angle of orientation (θ). As used herein, the term “target body” refers to the object to be imaged, which includes a patient, a particular portion of a patient, a phantom, exercised tissue, or any object of interest. In most cases, such target bodies are deformable target bodies.

In this disclosure, the use of the term “real-time” or “real time” refers to activities that take place within one session of imaging, diagnosis, or treatment of a patient. For example, “real-time” display of the target body refers to the ability to display the target body while the patient is being imaged by an ultrasound imaging system. For example, “real-time” therapy assessment refers to the ability to provide feedback of a particular treatment while a patient is still examined/treated by a physician.

In this disclosure, the phrase “maximum percent fill-in” is used to refer to when the axial-shear strain fill-in value approximates or attains a certain maximum value. The percent fill in represents the finite (non-zero) axial-shear strain values of the pixels inside the inclusion in an axial-shear strain elastogram (ASSE). This condition results from the axial-shear strain that occurs when the ASSE is obtained using a non-normal orientation of the axis of compression with regard to the long axis of the inhomogenity (inclusion or lesion). The presently disclosed method utilizes alterations in the angle of the compression which is exerted using the transducer to achieve/identify maximum percent fill-in.

### DETAILED DESCRIPTION

Presently described are methods and apparatus for generating and analyzing ASSE such that a readily interpreted signal is used to reliably and non-invasively identify the boundaries of an asymmetrical inhomogenity and further characterize the nature of the binding of the inhomogenity (for example, inclusion or lesion) and the surrounding environment, i.e. is it loosely or firmly bound. Furthermore in those instances where the inhomogeneity is a tumor within normal tissue these methods allow on to accurately and noninvasively obtain information regarding the malignant or benign nature of the tumor.

The disclosed methods also provide improved methods of reliably visualizing and identifying therapeutically induced tissue lesions, such as those created by HIFU treatment. A great benefit of the presently disclosed methods is the ease with which this information is obtained, presented and unambiguously interpreted.

In some embodiments, methods utilizing ASSE are described. One such method comprises generation of ASSE of a target body and identifying non-zero axial-shear strain on the ASSE to identify the boundaries of an inclusion in the target body. In another embodiment, ASSE of a target body and identification of regions of non-zero axial-shear strain on the ASSE is used to characterize the bonding of an inclusion to the target body as either loose or firm. In a further embodiment, when the inclusion is a tumor in tissue, ASSE of the tissue and identification of regions of non-zero axial-shear strain on the ASSE is used to characterize the likely hood that the tumor is benign or malignant. Provided in further embodiments, a method utilizing ASSE is described that can be used to provide direct and assess therapy in real time. The details of these methods and systems suitable for applying such methods are described in detail herein.

Ultrasound (US) elastography is a technique to image the stiffness variation in soft-tissues. This technique involves acquiring US (RF/envelope) signals from an imaging plane before and after the application of a small (e.g., ˜1%) quasi-static compression, which can be deformation exerted by an ultrasound transducer or caused momentarily by patient movement (breathing, etc). Typically, the pre- and post-compression frames are processed to generate images of local strain that are commonly known as elastograms. When the elastogram depicts axial strain values, it is referred to as an ASE. When the elastogram depicts axial-shear strain values, it is referred to as an ASSE.

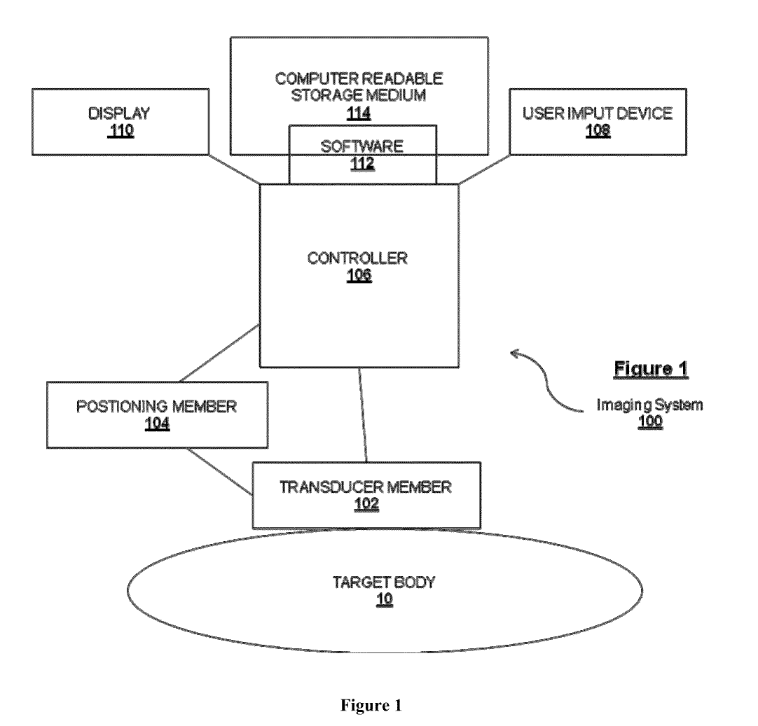

A schematic view of an exemplary imaging system 100 is shown in FIG. 1. Imaging system 100 includes a transducer member 102, a positioning member 104, a controller 106 operably coupled to the transducer member 102 and the positioning member 104, a user input device 108 (such as keyboard, trackball, mouse, joystick and/or touch screen), and a display 110.

Transducer member 102 comprises transducer elements and is operably linked to controller 106 through positioning member 104. However, transducer member 102 may be directly connected with controller 106. Positioning member 104 comprises a motor such that it can adjust position automatically and is configured to position transducer member 102 with 3 degrees of freedom, such that it can position the transducer member 102 linearly and angularly in all directions (preferably simultaneously) such that transducer member 102 remains in both mechanical and acoustic contact with and can compress target body 10 while emitting ultrasound energy and receiving echo signals from a multiplicity of angles.

To facilitate the ability of the transducer member 102 to move with 3 degrees of freedom, the transducer member 102 may be, for example, operably linked to positioning member 104 by a swivel or a parallel linkage.

Transducer member 102 is sonically coupled to a target body 10. Transducer member 102 is moved by positioning member 104 and controlled by controller 106 to provide imaging of at least a portion of a region of interest (ROI) of target body 10. ROI include but are not limited to areas that are believed to contain inclusions, areas that contain inclusions or areas that have received or require treatment, such as by HIFU.

Controller 106 comprises the circuitry known to those of ordinary skill in the elastography arts to generate and transmit analog pulses of ultrasound energy to transducer member 102, and to receive analog echo signals which are processed into digital echo signals and retrievably stored in a computer-readable storage medium (CRSM or digital storage device or memory) 114 that is used to store images for later viewing and/or processing and display data based upon the received and processed signals. Display 110 is capable of visually displaying strains, strain ratios, displacements, and slopes that are determined and/or estimated using various embodiments of the present invention, described herein.

In one embodiment, controller 106 is configured to execute one or more of the methods discussed herein. In one embodiment, at least a portion of each method executed by controller 106 is provided as a portion of software 112. In other embodiments, controller 106 also controls a HIFU System such as, but not limited to, those disclosed in: U.S. Pat. No. 4,084,582; U.S. Pat. No. 4,207,901; U.S. Pat. No. 4,223,560; U.S. Pat. No. 4,227,417; U.S. Pat. No. 4,248,090; U.S. Pat. No. 4,257,271; U.S. Pat. No. 4,317,370; U.S. Pat. No. 4,325,381; U.S. Pat. No. 4,586,512; U.S. Pat. No. 4,620,546; U.S. Pat. No. 4,658,828; U.S. Pat. No. 4,664,121; U.S. Pat. No. 4,858,613; U.S. Pat. No. 4,951,653; U.S. Pat. No. 4,955,365; U.S. Pat. No. 5,036,855; U.S. Pat. No. 5,054,470; U.S. Pat. No. 5,080,102; U.S. Pat. No. 5,117,832; U.S. Pat. No. 5,149,319; U.S. Pat. No. 5,215,680; U.S. Pat. No. 5,219,401; U.S. Pat. No. 5,247,935; U.S. Pat. No. 5,295,484; U.S. Pat. No. 5,316,000; U.S. Pat. No. 5,391,197; U.S. Pat. No. 5,409,006; U.S. Pat. No. 5,443,069, U.S. Pat. No. 5,470,350, U.S. Pat. No. 5,492,126; U.S. Pat. No. 5,573,497, U.S. Pat. No. 5,601,526; U.S. Pat. No. 5,620,479; U.S. Pat. No. 5,630,837; U.S. Pat. No. 5,643,179; U.S. Pat. No. 5,676,692; U.S. Pat. No. 5,840,031; U.S. Pat. No. 5,762,066; U.S. Pat. No. 6,685,640; US Patent Publication Nos: 2009/0069677 and 2010/0036291.

In some embodiments of the imaging system 100, the controller 106 comprises a processor and the CRSM 114 contains the software 112 that when executed by the processor, causes the imaging system 100 to perform the methods as described herein. In various embodiments, the controller further comprises, a scan generator, amplifier, signal receiver(s) and scan converter and processor.

In other embodiments, an imaging system comprises a transducer or a transducer array, which emits ultrasound pulses and receives/detects the returning echoes, an amplifier that amplifies the detected echo signal, a scan generator that produces pulse sequences and scan converter that transforms analog signals to digital signals. The imaging system may further comprise a processor that processes and computes the transformed signal to generate images and a display that displays the generated images, alternative forms of report are also envisioned (such as but not limited to a numerical scale, a printout, etc.).

The transducer member 102 of the imaging system 100 capable of performing the methods described herein comprises the ability to produce ultrasound pulses and receives/detects the returning echoes. In some embodiments the transducer member 102 also contains position sensors. In some embodiments, the controller produces pulse sequences for the obtaining of sonograms, ASEs and ASSEs.

In another embodiment, CRSM 114 contains software that, when executed by a processor, causes the processor to generate an ASSE of a target body; and identify the presence of non-zero axial-shear strain on the ASSE, wherein the non-zero axial-shear strain at least partially outlines an inclusion; and identifies the presence or absence of non-zero axial-shear strain inside the inclusion on the ASSE. In a further embodiment, an imaging system comprises a processor and a CRSM 114 containing software 112 that, when executed by the processor, causes the imaging system to generate an ASSE of a target body; identify the presence of non-zero axial-shear strain on the ASSE, wherein the non-zero axial-shear strain at least partially outlines an inclusion; and identify the presence or absence of non-zero axial-shear strain inside the inclusion on the ASSE. In an embodiment, such CRSM 114 and/or imaging system 100 are configured to diagnose inhomogeneities/inclusions in the target body. In an embodiment, such CRSM 114 and/or imaging system are configured to classify malignant and benign tumors in the target body.

In a further embodiment, CRSM 114 contains software 112 that, when executed by a processor, causes the processor to: generate an ASE of a target body; generate an ASSE of said target body; identify the presence of non-zero axial-shear strain on the ASSE and display percent fill-in; compute and display the maximum percent fill-in either as an ASSE or other image, numerically on a predetermined scale or in any way in which users could quickly interpret the result; identify the boundaries of the inclusions or lesions utilizing said non-zero axial-shear strain; combine the inclusion boundaries obtained from ASE and ASSE to obtain the complete boundaries of said one or more lesions.

In a still further embodiment, the software 112 when executed by a processor, causes the processor to generate a better defined ASSE of said target body by varying the compression axis orientation versus the inclusion's axis and combining the images to get a better-defined contour of the inclusion.

In yet another embodiment, an imaging system 100 contains a processor and a CRSM 114 containing software that, when executed by the processor, causes the imaging system 100 to generate an ASE of said target body and an ASSE of said target body; identify the presence of non-zero axial-shear strain on the ASSE; identify parts of the boundaries of said inclusion or lesion utilizing said non-zero axial-shear strain and combine the inclusion boundaries obtained from ASE and ASSE to obtain the complete boundaries of said one or more lesions.

In a further embodiment, CRSM 114 contains software 112 that, when executed by a processor, causes the processor to generate an generate an ASSE of the target body; identify the presence of non-zero axial-shear strain on the ASSE; and refine the boundaries of the one or more inclusions utilizing the non-zero axial-shear strain method disclosed.

In yet another embodiment, imaging system 100 contains a processor and a CRSM 114 containing software that, when executed by the processor, causes the imaging system 100 to generate an ASE of a target body, wherein the ASE comprises one or more inclusions having boundaries; generate an ASSE of the target body; identify the presence of non-zero axial-shear strain on the ASSE; and refine the boundaries of the one or more inclusions utilizing the non-zero axial-shear strain.

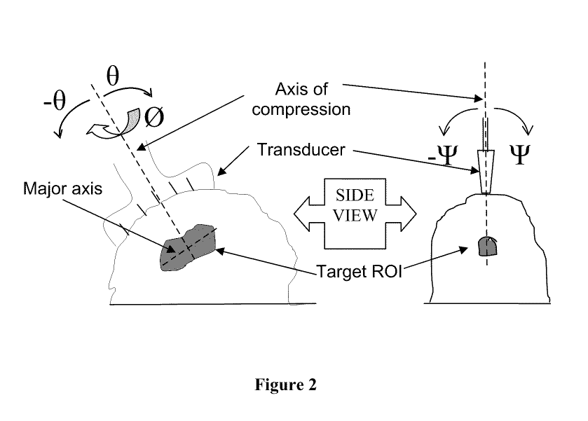

FIG. 2 is a schematic representation of the set-up using a cross-section view showing different relative orientations between the transducer and inhomogeneity coordinate systems: Pitch angle (θ), Yaw angle (ø) and roll angle (Ψ).

Traditional ultrasonic diagnosis is achieved by transmitting ultrasonic energy into a target body and generating an image from the resulting echo signals. A transducer is used to both transmit the ultrasonic energy and to receive the echo signals. During transmission, the transducer converts electrical energy into mechanical vibrations. Acquired echo signals produce mechanical oscillations in the transducer which are reconverted into electrical signals for amplification and recognition. A plot or display (e.g., on an oscilloscope, etc.) of the electrical signal amplitude versus echo arrival time yields an amplitude line (A-line) or echo sequence corresponding to a particular ultrasonic transmission. When the A-line is displayed directly as a modulated sinusoidal pattern at radio frequency (“RF”), it is typically referred to as an RF or “undetected” signal. For imaging, the A-line is often demodulated to a non-RF or “detected” signal.

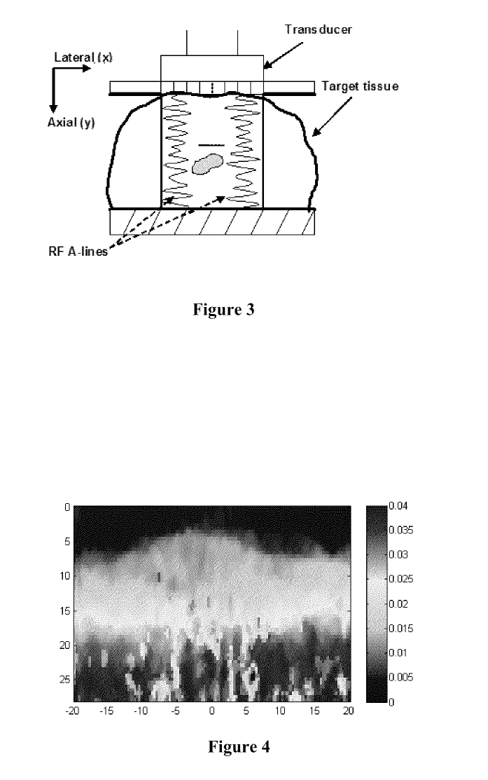

In a process that has become known as elastography (described, for example, in U.S. Pat. No. 5,107,837), RF A-lines are obtained from the target tissue before and after a slight compression (along the y direction) and are referred to as pre- and post-compression RF signals (or pre- and post RF frames) as shown in FIG. 3.

The pre- and post-compression RF signals are then subjected to the following signal processing. RF signal space is segmented into small overlapping windows and the corresponding RF windows from pre- and post compression frames are subjected to correlation-based displacement tracking algorithm to obtain axial and lateral displacement maps. A typical example of axial displacement map is shown in FIG. 4. It is known that the lateral (uxy) displacement map is very noisy and is not used by itself.

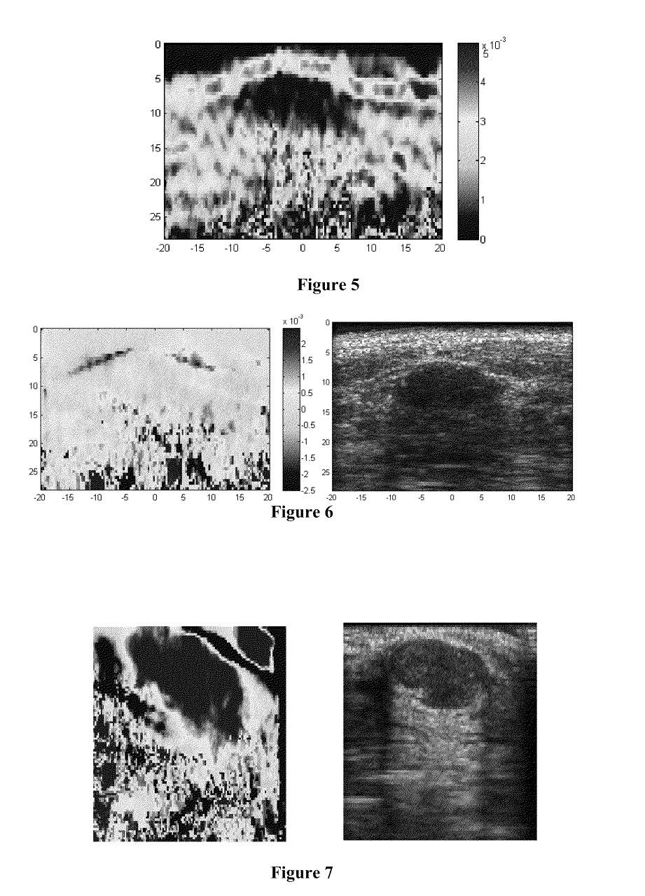

Traditionally, the axial-strain is computed as the gradient of the axial displacement along the y-direction (axial) and the lateral-strain is computed as gradient of lateral displacement along x-direction (lateral). A typical axial strain elastogram is shown in FIG. 5. This image is obtained by computing the axial gradient of FIG. 6.

Good quality images were obtained by taking the gradient of the axial displacement along x-direction (lateral) this is known as axial-shear strain elastogram (as described for example, ThitaiKumar A, Krouskop T A, Garra B S and Ophir J. Visualization of bonding at an inclusion boundary using axial-shear strain elastography: a feasibility study. Physics in Medicine and Biology 52, 2615-2633, 2007). This constitutes one component of the total shear strain which is defined as an average of axial-shear strain and lateral-shear strain (gradient of lateral displacement along y-direction). This estimator is less noisy than the simple gradient estimator.

Until the presently described methods were available visual feedback to localize inhomogeneous inclusions such as tumors using sonography and elastography was obtained from B-mode images in real-time. Often the shape and orientation of tumor is inferred from the B-mode by a sonographer and thus it is somewhat subjective and reliable interpretations are dependent upon the experience and abilities of the operator.

To facilitate a comparison, the images obtained in a typical B-mode and ASSE, images obtained from a pathologically-confirmed benign tumor are shown in FIG. 7. As shown in the ASSE on the left, the inventors noted that there was a “thin-high intensity” axial-shear strain zone near the tumor boundary, but none inside the tumor itself. In part, a similar observation inspired the inventors to develop embodiments of the novel methods of the disclosure that provide methods of analyzing ASSE allowing one to clearly identify the margins of an inclusion and determine if the inclusion were loosely or firmly bonded to the surrounding environment. These methods were then extended to encompass analysis of multiple tumors and further developed and refined using model systems such that it was determined that the orientation of the axis of the compression must be non-normal with regards to the long axis of the inclusion. In some embodiments the present disclosure provides methods by which such parameter are considered and also provides methods to automate the process, such that the required data can be obtained reliably, processed and interpreted with confidence.

To detect high intensity axial-shear strain zones at the boundaries of, and within, inhomogeneities or inclusions (such as, but not limited to, tumors) and the surrounding environment (tissue), a parameter described herein as percent fill-in of a tumor image in an ASSE image has been developed. The determination of a maximum percent fill-in allows the clear identification of the margins of an inclusion and characterizes the degree of binding, is it loosely or firmly bonded to the surrounding environment. Some embodiments of the present disclosure provide that high maximum percent fill-in of ASSE is indicative of the presence of a benign tumor, while low levels of maximum percent fill-in is indicative of a malignancy.

Inhomogeneities include all types of inclusions such as, but not limited to, lesions, malignant tumors or benign tumors. Therefore, the disclosed method may be used to diagnose diseases, such as breast cancer, prostate cancer and other cancers. In some embodiments, lesions may be intentional such as those created by therapies with HIFU.

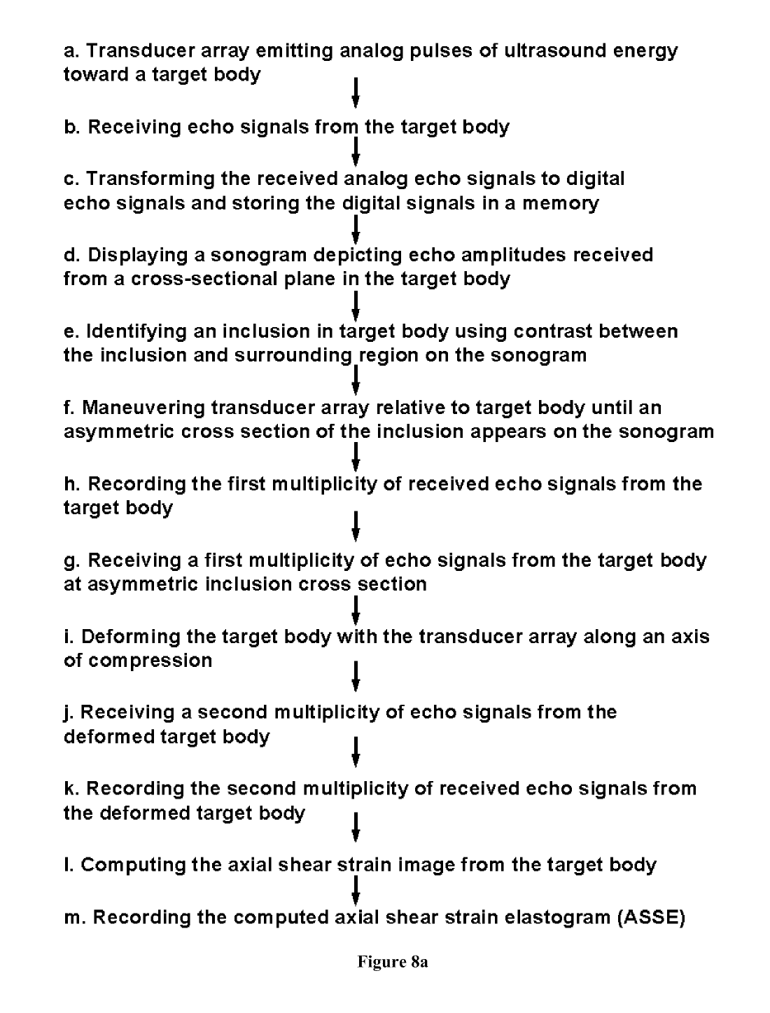

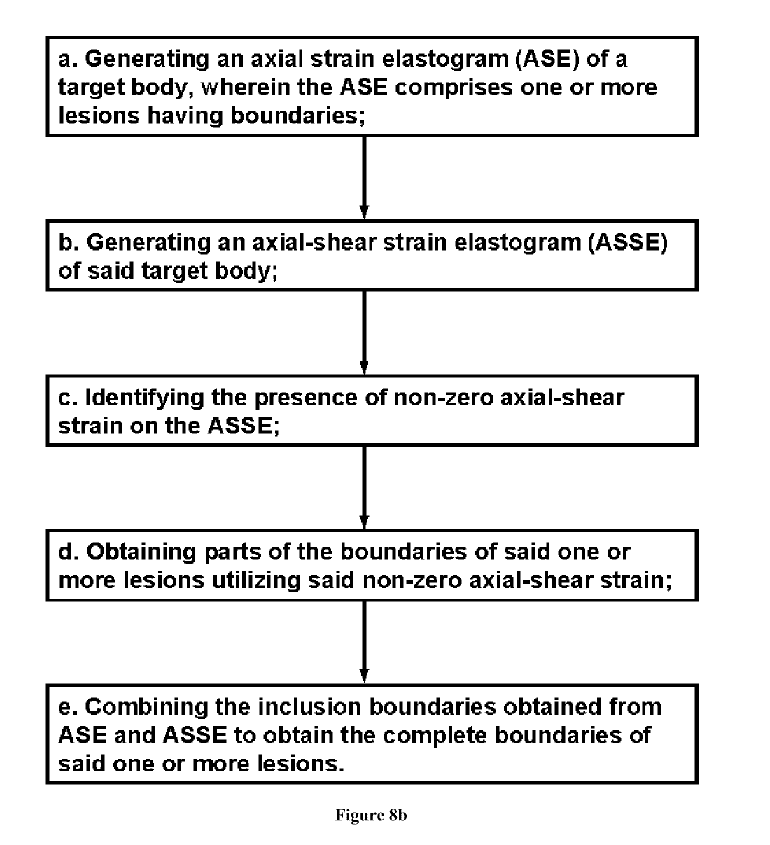

As illustrated in FIG. 8a, in one embodiment is a method for imaging axial shear strain properties of inhomogeneities or asymmetric inclusions comprising the following steps: (a) positioning an energized ultrasonic transducer array, comprising groups of transducer elements on the surface of a deformable target body so as to obtain acoustic and mechanical contact between the transducer array and the target body, the transducer array emitting analog pulses of ultrasound energy at a rate of at least ten frames per second; (b) receiving echo signals from the target body; (c) transforming the received analog echo signals to digital echo signals and retrievably storing the digital signals in a memory; (d) displaying a sonogram depicting echo amplitudes received from a cross-sectional plane in the target body using at least a portion of the digital echo signals; (e) identifying an inclusion in the target body using contrast between the inclusion and its surrounding region on the sonogram; (f) maneuvering the transducer array with respect to the target body until an asymmetric cross section of the inclusion appears on the sonogram; (g) receiving a first multiplicity of echo signals from the target body at asymmetric inclusion cross section; (h) recording the first multiplicity of received echo signals from the target body; (i) deforming the target body with the transducer array along an axis of compression by an amount in the range of from 0.1% to 5% of the thickness of the target body along the axis of compression; (j) receiving a second multiplicity of echo signals from the deformed target body; (k) recording the second multiplicity of received echo signals from the deformed target body; (l) computing the axial shear strain image from the target body; and (m) recording the computed ASSE. An asymmetric cross section of the inclusion on a sonogram is obtained when 0≠0° or 90°.

In another embodiment is a method for imaging axial shear strain properties of inhomogeneities or asymmetric inclusions comprising the following steps: (a) positioning an energized ultrasonic transducer array, comprising groups of transducer elements on the surface of a deformable target body so as to obtain acoustic and mechanical contact between the transducer array and the target body, the transducer array emitting analog pulses of ultrasound energy at a rate of at least ten frames per second; (b) receiving echo signals from the target body; (c) transforming the received analog echo signals to digital echo signals and retrievably storing the digital signals in a computer-readable storage medium; (d) displaying a sonogram depicting echo amplitudes received from a cross-sectional plane of the target body using at least a portion of the digital echo signals; (e) identifying an inclusion in the target body using contrast between the inclusion and its surrounding region on the sonogram; (f) maneuvering the transducer array with respect to the target body until an asymmetric cross section of the inclusion appears on a sonogram; (g) orienting the transducer array at multiple pitch angles with respect to the long axis of the inhomogeneity; (h) receiving a multiplicity of echo signals from the target body at each of the pitch angles; (i) recording the multiplicity of received echo signals from the target body; (j) deforming the target body with the transducer array along an axis of compression by an amount in the range of 0.1% to 5% of the thickness of the target body along the axis of compression; (k) receiving a multiplicity of echo signals from the deformed target body; (l) recording the multiplicity of received echo signals from the deformed target body; (m) computing the axial shear strain image from the target body; and (n) recording the computed axial shear strain image.

In another embodiment, a method to determine bonding conditions at the inhomogeneity boundary using axial-shear strain distribution comprising the following steps: (a) positioning an energized ultrasonic transducer array, comprising groups of transducer elements on the surface of a deformable target body so as to obtain acoustic and mechanical contact between the transducer array ant the target body, said transducer array emitting analog pulses of ultrasound energy at a rate of at least ten frames per second; (b) receiving echo signals from the target body; (c) transforming the received analog echo signals to digital echo signals and retrievably storing said digital signals in a computer-readable storage medium; (d) displaying a sonogram depicting echo amplitudes received from a cross-sectional plane in the target body using at least a portion of said digital echo signals; (e) identifying an inhomogeneity comprising a long axis in the target body using contrast between the inhomogeneity and its surrounding region on the sonogram; (f) maneuvering the transducer array with respect to the target body until a cross section of the inhomogeneity appears on the sonogram; (g) orienting the transducer array at several yaw angles with respect to the long axis of the inhomogeneity; (h) receiving a multiplicity of echo signals from the target body at each of said yaw angles; (i) recording the multiplicity of received echo signals from the target body; (j) deforming the target body with the transducer array along an axis of compression by an amount in the ranges from 0.1% to 15% (for example, about 0.1%, 0.5%, 1%, 2%, 3%, 4%, 5%, 6%, 7%, 8%, 9%, 10%, 12%, 15%) of the thickness of the target body along the axis of compression; (k) receiving a multiplicity of echo signals from the deformed target body; (l) recording the multiplicity of received echo signals from the deformed target body; (m) computing the axial shear strain image from the target body; and (n) recording the computed axial shear strain image.

In the presently disclosed method, the direction of compression is directed using repeated relocation of the transducer to compress the tissue and to obtain asymmetric cross-section of the inclusion. In doing so these methods yield images of the underlying axial-shear strain due to specific direction of compression, wherein the directions of the US beam and compression both originate from the transducer. The instant method results in a more reliable interpretation of the bonding condition of the inclusion, than would other methods such as those of U.S. Pat. No. 7,331,926, entitled “Ultrasonic Elastography Providing Axial, Orthogonal, And Shear Strain” in which the US beam itself is deliberately steered at several angles with regards to the axis of compression. The presently disclosed methods will image bonding properties reliably and accurately. No fill-in effect is observed when using total shear strain, defined as the sum of axial-shear strain and lateral-shear strain. Although the inventors could obtain a fill-in effect using lateral shear strain, the quality of the information was unacceptably inferior because as established it is noisy and cannot be used in practice. Somewhat surprisingly, this disclosure establishes that axial-shear strain information provides a practical method of establishing the fill-in of the inclusion and a more accurate interpretation of bonding properties.

In a further embodiment, a method is disclose that can be used to determine bonding conditions at inhomogeneity boundary using axial-shear strain distribution comprising of: (a) positioning an energized ultrasonic transducer array, comprising groups of transducer elements on the surface of a deformable target body so as to obtain acoustic and mechanical contact between the transducer array ant the target body, said transducer array emitting analog pulses of ultrasound energy at a rate of at least ten frames per second; (b) receiving echo signals from the target body; (c) transforming the received analog echo signals to digital echo signals and retrievably storing said digital signals in a memory; (d) displaying a sonogram depicting echo amplitudes received from a cross-sectional plane in the target body using at least a portion of said digital echo signals; (e) identifying an inhomogeneity comprising a long axis in the target body using contrast between the inhomogeneity and its surrounding region on the sonogram; (f) maneuvering the transducer array with respect to the target body until a cross section of the inhomogeneity appears on the sonogram; (g) orient the transducer array at several roll angles with respect to the long axis of the inhomogeneity (h) receiving a multiplicity of echo signals from the target body at each of said roll angles; (i) recording the multiplicity of received echo signals from the target body; (j) deforming the target body with the transducer array along an axis of compression by an amount in the range of 0.1% to 5% of the thickness of the target body along the axis of compression; (k) receiving a multiplicity of echo signals from the deformed target body; (l) recording the multiplicity of received echo signals from the deformed target body; (m) computing the axial shear strain image from the target body; and (n) recording the computed axial shear strain image. An asymmetric cross section of the inclusion on a sonogram is obtained when 0≠0° or 90°.

In some embodiments, the inclusion is a lesion or a tumor. In an embodiment, identifying the inclusion in the target body comprises using strain contrast of inclusion with respect to its surrounding on an ASE of the target body. In some embodiments, the deformation of target body may be coincident to the motions that momentarily accompany patient movement (breathing, etc.). In another embodiment, the method further comprises recording an axial displacement image.

In other embodiments, the generation of an ASSE comprises: a) position an energized ultrasonic transducer or transducer array (e.g., a linear array) on the surface of a deformable target body so as to obtain a good acoustic and mechanical contact; b) transmit a multiplicity of pulses along the array; c) receive echo signals from the deformable target body; d) record the multiplicity of echo signals from the deformable target body; e) deform the deformable body target body by a small amount; f) receive echo signals from the deformable target body; g) record the multiplicity of echo signals from the deformed deformable target body; h) compute and record the axial shear strain image from the deformable target body; and i) compute and record the axial strain elastogram from the deformable target body. In some embodiments, some or all of these steps are repeated until one a maximum percent fill-in ASSE is obtained.

In some embodiments, the deformable target body is biological tissue. In some embodiments, the deformable target body is a male prostate. In some embodiments, the deformable target body is a female breast. In some embodiments, the deformable target body is a thyroid. In some embodiments, the deformable target body is a liver. In some embodiments, the target body is a uterus. In some embodiments, the target body is a cervix. In some embodiments, the target body is a kidney. In some embodiments, the target body is a pancreas. In some embodiments, the target body is brain. In some embodiments, the target body is heart. In some embodiments, the target body is muscle.

Yet another disclosed embodiment is a method for imaging axial shear strain properties of inhomogeneities or asymmetric inclusions that comprises the following steps: (a) a transducer, that emits analog pulses of ultrasound energy at a rate of at least ten frames per second, is positioned in contact with the surface of a deformable target body so as to obtain acoustic and mechanical contact between the transducer and the target body; (b) the target body is deformed with the transducer along an axis of compression; (c) a first multiplicity of analog echo signals from the target body are (d) transformed to digital echo signals that are retrievably stored in a computer-readable storage medium; (e) the ASSE is computed and the percent fill-in is determined using the methods described in this disclosure; (f) this information is retrievably stored in the computer-readable storage medium; (g) the transducer is reoriented on the target body and relative to inclusion and the process is repeated through a minimum of three yaw angles, until which time a maximum percent fill-in has been established; (h) the maximum percent fill-in is displayed either as an image or assigned a value on a predetermined scale. If the maximum fill-in is high it is an indication that the inclusion is loosely bound to the surroundings. If the maximum fill-in is low it is an indication that the inclusion is firmly bound to the surroundings. If the inclusion is a tumor, a high maximum fill-in is indicative of a benign lesion but if the maximum fill-in is low it is indicative of a malignant cancer, thus in several embodiment the present disclosure provide a method of determining the risk of malignancy associated with an inclusion.

The determination of a maximum percent fill-in allows the clear identification of the margins of an inclusion and characterizes the degree of binding, is it loosely or firmly bonded to the surrounding environment. Some embodiments provide that high maximum percent fill-in of ASSE are indicative of the presence of a benign tumor, while low levels of maximum percent fill-in are indicative of a malignancy.

In one embodiment, methods for using axial shear strain properties and percent fill-in of an asymmetric inhomogeneity to identify the boundaries of the inhomogeneity. In another embodiment, methods for using axial shear strain properties and percent fill-in of an asymmetric inhomogeneity to characterize the binding of the inhomogeneity and its surround. In another embodiment, methods for using axial shear strain properties and percent fill-in of an asymmetric tumor to determine if it is benign or malignant. In another embodiment, methods for using axial shear strain properties of an asymmetric inclusion and percent fill-in to identify the boundaries within which a therapy (such as HIFU) should be directed. In another embodiment, methods for using axial shear strain properties of an asymmetric inclusion and percent fill-in to identify regions of untreated tissue that lie between regions of treated tissue. In another embodiment, methods for using axial shear strain properties of an asymmetric inclusion and percent fill-in to identify regions of untreated tissue that lie between regions of treated tissue. In another embodiment, methods for using axial shear strain properties of an asymmetric inclusion and percent fill-in to identify regions of untreated tissue that lie between regions of treated tissue. In another embodiment, further comprising methods wherein the identified untreated regions are treated and the process is repeated until the entire lesion has been treated. In another embodiment, the asymmetric inhomogeneity is an inclusion, lesion or tumor.

In another embodiment, a method for characterizing the bonding properties of an inclusion using ASSE, said method comprising: (a) positioning an energized ultrasonic transducer on the surface of a deformable target body so as to obtain acoustic and mechanical contact between the transducer and the target body, said transducer emitting analog pulses of ultrasound energy at a rate of at least five frames per second; (b) receiving echo signals from the target body; (c) transforming the received analog echo signals to digital echo signals and retrievably storing said digital signals in a memory (CSRM); (d) maneuvering the transducer array with respect to the target body until an asymmetric cross section of the inclusion is identified; (e) receiving a multiplicity of echo signals from the target body; (f) recording the multiplicity of received echo signals from the target body; (g) deforming the target body with the transducer array along an axis of compression by an amount in the range of 0.1% to 5% of the thickness of the target body along the axis of compression; (h) receiving a multiplicity of echo signals from the deformed target body; (i) recording the multiplicity of received echo signals from the deformed target body; (j) computing and displaying the axial shear strain image from the target body; (k) recording a computed ASSE; (l) identifying the presence of non-zero axial-shear strain on the ASSE, wherein said non-zero axial-shear strain outlines an inclusion; (m) identifying the presence or absence of non-zero axial-shear strain inside the inclusion on the ASSE; (n) quantifying the non-zero axial-shear strain region inside the identified inclusion on the ASSE as a percent fill-in value; (o) providing the normalized percent fill-in value as a visual feedback to guide the operator to repeat steps (d) through (n) at a minimum of three different asymmetric orientations of the inclusion.

In another embodiment, the method above wherein (d) identification of the inclusion is accomplished using ASE or sonography (in which the images are displayed on a sonogram depicting echo amplitudes received from a cross-sectional plane in the target body using at least a portion of said digital echo signals). In another embodiment, the method above further comprises recording the axial displacement image on a CRSM. In another embodiment, the method above wherein the presence of non-zero axial-shear strains partially outlines the inclusion on the ASSE suggesting the presence of an inclusion inside said target body. In another embodiment, the method above wherein the presence of non-zero axial-shear strain inside the inclusion on the ASSE suggests the presence of loosely-bonded inclusion inside said target body. In another embodiment, the method above wherein the absence of non-zero axial-shear strain inside the inclusion on the ASSE suggests the presence of firmly-bonded inclusion inside said target body. In another embodiment, the method above used to classify malignant and benign tumors. In another embodiment, the method above wherein said target body is a female breast, a male prostate, a thyroid, a liver, uterus, cervix, kidney, pancreas, brain, muscle, heart or other organ. In another embodiment, the described methods can be applied when the inclusion is a lesion. An imaging system containing software that, when executed by a processor, causes the processor to perform the methods described.

In another embodiment a method of identifying the bonding characteristics that exist between a deformable target body and an asymmetric inclusion within it, said method comprising: (a) positioning an energized ultrasonic transducer on the surface of the target body so as to obtain acoustic and mechanical contact between the transducer and the target body said transducer emitting analog pulses of ultrasound energy at a rate of at least five frames per second; (b) deforming the target body with the transducer along an axis of compression; (c) receiving a multiplicity of echo signals from the deformed target body; (d) recording the received echo signals from the deformed target body retrievably in a computer-readable storage medium; (e) generating and displaying an axial shear strain image containing the inclusion; (f) identifying the presence of non-zero axial-shear strain within the inclusion and its surroundings; (g) computing the non-zero axial-shear strain that is in the inclusion as a percent fill-in value; (h) displaying the percent fill-in value. In another embodiment, this method further comprises: (i) repeating steps a-h, after systematically changing the angle of compression relative to the inclusion.

In an alternative embodiment, this method wherein (i) comprises translation of the transducer relative to the inclusion or manipulating the transducer through a minimum of three yaw angles relative to the inclusion. In another embodiment, this method further comprises (j) charting percent fill-in and the angle of compression relative to the inclusion. In another embodiment, this method further comprises (k) using the chart to identify the maximum percent fill-in. When the deformable target body is normal tissue, the asymmetric inclusion is a tumor with a maximum percent fill-in of less than 20% this result raises a high suspicion of malignancy. When the tumor has a maximum percent fill-in of between 20% to 50%, the result raises a moderate suspicion of malignancy. When the tumor has a maximum percent fill-in of between 50% to 80%, the result does not suggest malignancy and when the tumor has a maximum percent fill-in greater than 80% it is very likely benign.

In another embodiment, a method of identifying the boundaries of an asymmetric inclusion contained within a deformable target body, said method comprising: (a) positioning an energized ultrasonic transducer on the surface of the target body so as to obtain acoustic and mechanical contact between the transducer and the target body said transducer emitting analog pulses of ultrasound energy at a rate of at least five frames per second; (b) deforming the target body with the transducer along an axis of compression; (c) receiving a multiplicity of echo signals from the deformed target body; (d) recording the received echo signals from the deformed target body retrievably in a computer-readable storage medium; (e) generating and displaying an axial shear strain image containing the inclusion; (f) identifying the presence of non-zero axial-shear strain within the inclusion and its surroundings; (g) computing the non-zero axial-shear strain that is within the inclusion and its surroundings as a percent fill-in value; (h) using the differential in percent fill-in of the inclusion compared with its surroundings to reliably identify the boundaries of the inclusion. In another embodiment, this method further comprises (i) after systematically changing the angle of compression relative to the inclusion, repeating steps a-h; and (j) combining this information to create an overlapping image to more accurately identify the boundaries of the inclusion. In other embodiments, the deformable target body is normal tissue, the asymmetric inclusion is a tumor and the boundary is the tumor margin.

HIFU lesions are tightly bound to the surrounding tissue thus when a loosely bound region is identified between tightly bound regions it represents a region of the target tissue that received insufficient HIFU therapy and should be retreated for maximum effect. Therefore in another embodiment, the above method can be used to (k) identify untreated regions between HIFU lesions by using the canceling properties of percent fill-in. In another embodiment, once identified untreated regions between HIFU lesions by using the canceling properties of percent fill-in; and these regions are (l) treated with HIFU.

In another embodiment a method of identifying the bonding characteristics that exist between a deformable target body and an asymmetric inclusion within it, said method comprising: positioning an energized ultrasonic transducer on the surface of the target body so as to obtain acoustic and mechanical contact between the transducer and the target body said transducer emitting analog pulses of ultrasound energy at a rate of at least five frames per second; deforming the target body with the transducer along an axis of compression; receiving a multiplicity of echo signals from the deformed target body; recording the received echo signals from the deformed target body retrievably in a computer-readable storage medium; generating and displaying an axial shear strain image containing the inclusion; identifying the presence of non-zero axial-shear strain within the inclusion and its surroundings; computing the non-zero axial-shear strain that is in the inclusion as a percent fill-in value; displaying the percent fill-in value.

In another embodiment this method further comprising after systematically changing the angle of compression relative to the inclusion repeating steps a-h. In another embodiment, these methods can comprise changing the angle of compression by translation of the transducer relative to the inclusion or by manipulating the transducer through a minimum of three yaw angles relative to the inclusion. In another embodiment these methods can further comprise charting percent fill-in and the angle of compression relative to the inclusion. In another embodiment these methods can further comprise using the chart to identify the maximum percent fill-in. In other embodiments the deformable target body is normal tissue, the asymmetric inclusion is a tumor and a maximum percent fill-in of less than 20% suggests malignancy and a maximum percent fill-in of greater than 80% suggests a benign tumor.

In another embodiment, a method of identifying the boundaries of an asymmetric inclusion contained within a deformable target body, said method comprising (a) positioning an energized ultrasonic transducer on the surface of the target body so as to obtain acoustic and mechanical contact between the transducer and the target body said transducer emitting analog pulses of ultrasound energy at a rate of at least five frames per second; (b) deforming the target body with the transducer along an axis of compression; (c) receiving a multiplicity of echo signals from the deformed target body; (d) recording the received echo signals from the deformed target body retrievably in a computer-readable storage medium; (e) generating and displaying an axial shear strain image containing the inclusion; (f) identifying the presence of non-zero axial-shear strain within the inclusion and its surroundings; (g) computing the non-zero axial-shear strain that is within the inclusion and its surroundings as a percent fill-in value; (h) using the differential in percent fill-in of the inclusion compared with its surroundings to reliably identify the boundaries of the inclusion. In another embodiment this method can further comprise: (i) after systematically changing the angle of compression relative to the inclusion, repeating steps a-h; and (j) combining this information to create an overlapping image to more accurately identify the boundaries of the inclusion. In some embodiments, the deformable target body is normal tissue, the asymmetric inclusion is a tumor and the boundary is the tumor margin. In other embodiments these methods further comprise (k) identifying the untreated regions between HIFU lesions by using the canceling properties of percent fill-in.

In another embodiment, a method for identifying the bonding characteristics of an inclusion within the normal tissue of an individual, the method comprising: (a) producing ultrasound axial-shear strain elastography images of a target region in said individual including or suspected of including an inclusion; (b) identifying from said axial-shear strain elastography image the presence of non-zero axial-shear strain within an inclusion and its surrounding normal tissue in said target region; (c) calculating the non-zero axial-shear strain that is in the inclusion as a percent fill-in value, wherein a calculated percent fill-in value of less than 50% is indicative of an inclusion firmly bonded to its surrounding tissue and a percent fill-in value greater than 50% is indicative of an inclusion loosely bonded to its surrounding tissue. In some embodiments, the method further comprises (d) after systematically changing the angle of compression relative to the inclusion repeating steps a-c, and; (e) determining a maximum percent fill-in value for the inclusion. In some embodiments of the method, said systematically changing the angle of compression comprises translating the transducer relative to the inclusion or manipulating the transducer through a minimum of three yaw angles relative to the inclusion. In additional embodiments, the method further comprises (f) charting percent fill-in and the angle of compression relative to the inclusion.

In another embodiment a method of diagnosing an individual having or suspected of having a tumor, comprising: (a) producing an ultrasound axial-shear strain elastography image of a target region in said individual, said region including or suspected of including a tumor; (b) identifying from said axial-shear strain elastography image the presence of non-zero axial-shear strain within an inclusion and its surrounding tissue in said target region; (c) calculating the non-zero axial-shear strain that is in the inclusion as a percent fill-in value, (d) systematically changing the angle of compression relative to the tumor and repeating steps a-c, (e) determining a maximum percent fill-in value for the tumor. In embodiments of the method a maximum percent fill-in value of less than 20% is suggestive of a malignant tumor. A maximum percent fill-in value between 20% to 50% is less suggestive of a malignant tumor as compared to a percent fill-in value of less than 20%. A maximum percent fill-in value of between 50% to 80% is suggestive of a benign tumor. A maximum percent fill-in value of greater 80% is more suggestive of a benign tumor as compared to a percent fill-in value between 50% to 80%.

In other embodiments, a method of accurately identifying the margins of a tumor within normal tissue, comprising: (a) producing an ultrasound axial-shear strain elastography image of a target region in said individual, said region including or suspected of including a tumor; (b) identifying from said axial-shear strain elastography image the presence of non-zero axial-shear strain within an inclusion and its surrounding tissue in said target region; (c) calculating the non-zero axial-shear strain that is in the inclusion, as a percent fill-in value; (d) systematically changing the angle of compression relative to the tumor and repeating steps a-c; (e) combining the resulting calculations to create an overlapping image that accurately identifies the boundaries of the tumor; and (f) displaying said image. In some embodiments, a method of directing tumor therapy comprising performing the method on an individual during tumor therapy, in real-time. In other embodiments said tumor therapy is HIFU.

Because HIFU lesions are tightly bound to the surrounding tissue (normal or tumor) there is little to no non-zero axial-shear strain within the lesion itself and therefore percent fill-in value ranges from background noise (10%) to almost 0%. Such tightly bound inclusions can be visualized using ASSE and the presently disclosed methods because these lesions produce a characteristic positive (represented by colors from green to red signal) and negative (represented by colors from green to blue) axial-shear strain pattern at the lesion boundary clearly identifies the lesion or multiple separated lesions within the ASSE of a tumor. Thus in some embodiments, a method of accurately identifying the margins of HIFU lesions in tumors is provided using ASSE wherein a characteristic 2 positive (represented by colors from green to red signal) and 2 negative (represented by colors from green to blue) axial-shear strain pattern at the lesion boundary that results from 2 positive (represented by colors from green to red signal) and 2 negative (represented by colors from green to blue) axial-shear strain patterns that alternate.

However, as the locations of individual lesions appear closer and closer to each other, the negative axial-shear strain region of one lesion cancels the proximal positive axial-shear strain region of the other, and one observes only 4 regions (2 per lesion) of finite axial-shear strain at the lesion boundary when one would have expected to see 8 regions, had the two lesions behaved independently. When untreated tissue is present within a HIFU treated tumor and the two HIFU lesions are separate it will result in the same ASSE pattern obtained with single HIFU lesions, thus providing the characteristic 8 regions (4 regions per lesion) pattern. But as newly generated HIFU lesions are formed within the untreated space between the two original lesions eventually the ASSE pattern will change to a 4 region pattern indicating that the lesion is not behaving as one lesion and previously untreated region is now treated.

The ability of ASSE to clearly identify HIFU lesions and to characterize untreated space are important utilities therefore in additional embodiments a method that can be used to identifying the region of untreated tumor tissue that is located between the boundary of two treated tumor tissue regions within a target region that comprises: (a) producing an ultrasound axial-shear strain elastography image of a target region in said individual, said region including or suspected of including a tumor region that contains at least a first HIFU lesion and a second HIFU lesion; (b) identifying in said axial-shear strain elastography image the presence of the first HIFU lesion and the second HIFU lesion by the presence of a characteristic (2 positive and 2 negative) axial-shear strain pattern located at the boundary of both the first HIFU lesion and a second HIFU lesion; (c) identifying in said axial-shear strain elastography image the untreated tumor tissue region that lies between the first HIFU lesion and a second HIFU lesion; (d) applying HIFU to the identified untreated tumor tissue region, until the boundary of the first HIFU lesion and the second HIFU lesion are united and boundary appears within an axial-shear strain elastography image with the characteristic (2 positive and 2 negative) axial-shear strain pattern that characterizes a single HIFU lesion. In another embodiment this method further comprises (e) systematically moving between HIFU lesions within the tumor region, and (f) repeating steps a-e until the entire tumor region has been treated. In other embodiments, a method of directing tumor therapy by identifying a region of untreated tumor tissue on an individual during tumor therapy, in real-time.

In a related embodiment a method that can be used to identifying the region of untreated tumor tissue that is located between the boundary of two treated tumor tissue regions within a target region that comprises: (a) producing an ultrasound axial-shear strain elastography image of a target region in said individual, said region including or suspected of including a tumor region that contains at least a first HIFU lesion and a second HIFU lesion; (b) identifying in said axial-shear strain elastography image the presence of the first HIFU lesion and the second HIFU lesion by the presence of a characteristic (2 positive and 2 negative) axial-shear strain pattern located at the boundary of both the first HIFU lesion and a second HIFU lesion; (c) identifying in said axial-shear strain elastography image the untreated tumor tissue region that lies between the first HIFU lesion and a second HIFU lesion; (d) repeatedly applying HIFU to the identified untreated tumor tissue region until the boundary of the first HIFU lesion and the second HIFU lesion are united and the boundary appears within a live-time axial shear strain elastography image as an axial shear strain pattern that is characteristic of a single HIFU lesion. In other embodiment this method further comprises (e) systematically moving between HIFU lesions within the tumor region, and (f) repeating steps a-e until the entire tumor region has been treated. In other embodiments, a method of directing tumor therapy by identifying a region of untreated tumor tissue on an individual during tumor therapy, live and in real-time.

In other embodiments, the disclosed methods and interpretations occur in real-time, while the patient is undergoing the procedure or the therapy. These methods therefore not only provide particularly accurate and useful results but they allow rapid implementation of the results in real-time, which not only improve the efficacy of a procedure but also the safety of it and make it more convenient and cost effective.

The presently disclosed methods reveal that when an inclusion is present within a deformable target body, axial shear strain will appear at the boundaries of the inclusion when the region in the target body comprising the inclusion is under compression and that ASSE provides a method of characterizing the interactions between the inclusion and the surroundings. It has been established that both asymmetry parameter (θ) and the aspect ratio (a/b) on the axial-shear strain will affect the degree of fill-in observed inside the inclusion. When the direction of compression used to generate the ASSE is non-normal or non-orthogonal (0≠0° or 90°) to an axis of the inclusion (if the inclusion is approximated by a shape with an axis), the presence of finite non-zero axial-shear strain values of the pixels inside the inclusion result in what is described herein as “fill-in”. Percent fill-in is a defined metric determined during ASSE analysis and which is calculated as the percentage of the inclusion's interior area that experienced axial-shear strain. The formula used to calculate percent fill-in is given in Eqn. 2 as described in Example 1 below. The presence of non-zero axial-shear strain inside the inclusion on the ASSE suggests the presence of loosely-bonded inhomogeneity inside the target body; and the absence of non-zero axial-shear strain inside the inclusion on the ASSE suggests the presence of firmly-bonded inhomogeneity inside the target body.

However, to accurately execute and interpret these methods the compression/deformation used to generate the ASSE must be non-normal or non-orthogonal (0@0° or 90°) to the axis of the inclusion in order to identify maximum non-zero axial-shear strain inside the inclusion that will appear as maximum fill-in. To assure that the ASSE is obtained using compression delivered in a non-normal or non-orthogonal to the axis of the inclusion a series of echo signals are collected from three or more positions along the target body and another ASSE is generated using compression/deformation exerted from a different angle. The steps for generating ASSE can be repeated until the compression/deformation occurs from an angle that is non-normal to the axis of the inclusion and maximum non-zero axial-shear strain inside the inclusion occurs and maximum fill-in is visualized.

The presence of significant non-zero axial-shear strain inside the inclusion on the ASSE indicates the presence of a loosely-bonded tumor and suggests the tumor is benign in nature, however, malignant tumors do not demonstrate significant level presence of non-zero axial-shear strain inside the inclusion on the ASSE. It should be noted that the noise in the system can result in a maximum percent fill-in of up to approximately 10%. Therefore, one can have high confidence that an inclusion that demonstrates only 10% maximum percent fill-in is tightly bound and is very likely invasive and thus malignant in nature. In some embodiments, non-zero axial-shear strain values fill more than 50% of the inclusion area. In some embodiments, non-zero axial-shear strain values fill more than 60% of the inclusion area. In some embodiments, non-zero axial-shear strain values fill more than 70% of the inclusion area. In some embodiments, non-zero axial-shear strain values fill more than 80% of the inclusion area. In some embodiments, non-zero axial-shear strain values fill more than 90% of the inclusion area.

In an embodiment, the generated ASSE is used in conjunction with an ultrasound sonogram or an ASE of the target body. In an embodiment, an ASE exhibits an inclusion area and an ASSE exhibits the boundaries of the inclusion; the ASSE and ASE are overlaid to more accurately depict the inclusion in the target body.

In a further embodiment, a method for identifying bonding property of an inclusion based on ASSE obtained via method as shown in FIG. 8a comprises a. identifying the presence of non-zero axial-shear strain on the ASSE, wherein the non-zero axial-shear strain at least partially outlines an inclusion; and b. identifying the presence or absence of non-zero axial-shear strain inside the inclusion on the ASSE. The presence of non-zero axial-shear strain at least partially outlining the inclusion on the ASSE suggests the presence of inclusion inside the target body; the presence of non-zero axial-shear strain inside the inclusion on the ASSE suggests the presence of loosely-bonded inclusion inside the target body; and the absence of non-zero axial-shear strain inside the inclusion on the ASSE suggests the presence of firmly-bonded inclusion inside the target body.

In an embodiment, a method is used to visualize the boundaries of a inclusion in a target body, the method comprising generating an ASSE of the target body; identifying the presence of non-zero axial-shear strain on the ASSE; and refining the boundaries of the one or more inclusions utilizing the non-zero axial-shear strain. In an embodiment, the method further comprises displaying the refined boundaries of one or more inclusions in real time. In some embodiments, the target body is biological tissue. In some embodiments, the target body is a male prostate. In some embodiments, the target body is a female breast. In some embodiments, the target body is a thyroid. In some embodiments, the target body is a liver.

In an embodiment, the method of visualizing one or more inclusions in the target body is used to provide real-time therapy assessment. In an embodiment, the method is used to visualize the boundaries of single or multiple lesions in the target body. In some embodiments, the method is able to visualize tissue between multiple lesions in the target body.

HIFU treatment of a large tumor is typically achieved by multiple small exposures that cover the entire volume. It is important to visualize the multiple smaller lesions and ensure that there remains no untreated region located between them. In an embodiment, the ASSE methods of the present disclosure are used to visualize the thin untreated regions and can be used to direct and monitor HIFU treatment.

In an embodiment, a method used to detect the presence of untreated regions of tissue among multiple HIFU-lesions using ASSE. The method comprises: a) position an energized ultrasonic transducer array on the surface of a deformable target body so as to obtain a good acoustic and mechanical contact; b) transmit a multiplicity of pulses along the array; c) receive echo signals from the deformable target body; d) record the multiplicity of echo signals from the deformable target body; e) deform the deformable body target body by a small amount (e.g., 1%); f) receive echo signals from the deformable target body; g) record the multiplicity of echo signals from the deformed deformable target body; h) compute and record the axial shear strain image from the deformable target body; i) compute and record the axial strain elastogram from the deformable target body; j) compute the boundary of the inhomogeneity based on axial shear strain distribution; k) refine the computed boundary using contrast of the inhomogeneity in axial strain elastogram; and l) observe the appearance of axial-shear strain zones among multiple HIFU-lesions.

In an embodiment, a method is used to visualize one or more inhomogeneities located within a region of interest (ROI) in a target body. The method comprises a) position an energized ultrasonic transducer or transducer array (e.g., a linear array) on the surface of a deformable target body so as to obtain a good acoustic and mechanical contact; b) transmit a multiplicity of pulses along the array; c) receive echo signals from the ROI of the deformable target body; d) record the multiplicity of echo signals from the ROI of the deformable target body; e) apply compression to deform the ROI of the deformable target body by a small amount; f) receive echo signals from the ROI of the deformable target body; g) record the multiplicity of echo signals from the deformed deformable target body; h) compute and record the axial shear strain image from the deformable target body; i) relocate the transducer array relative to the inhomogeneities through a minimum of three yaw angles and j) repeat steps e-j until the maximal percent fill-in is established; (i) display the maximal percent fill image or represent it numerically.

In an embodiment, the method further comprises overlaying ASSE on corresponding ASE. In some embodiments, the inhomogeneity is a lesion. In some embodiments, the lesion is created by HIFU. In some embodiments, the lesion is benign. In some embodiments, the lesion is malignant. In some embodiments, the deformation of the target body is caused by the transducer.

In an embodiment, as shown in FIG. 8b, a method for detecting HIFU-created lesions using ASSE, comprises: a. generating an ASSE of said target body; b. identifying the presence of non-zero axial-shear strain on the ASSE; c. identifying boundaries of said one or more lesions utilizing said non-zero axial-shear strain.

In some embodiments, the method further comprises displaying the boundaries of said one or more inclusions in real time. In some embodiments, the method further comprises displaying a composite image derived from ASSE and ASE of said target body. In some embodiments, the composite image is a color-overlay image. In some embodiments, the method is used to provide real-time HIFU-therapy monitoring. In some embodiments, the method is used to visualize the boundaries of single or multiple lesions in said target body. In some embodiments, the method is used to visualize untreated tissue between multiple lesions in said target body.

The following section provides further details regarding examples of various embodiments. It should be appreciated by those of skill in the art that the techniques disclosed in the examples that follow represent techniques and/or compositions discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention. These examples are illustrations of the methods and systems described herein and are not intended to limit the scope of the invention.

### EXAMPLES

Although the figures in this patent application appear in grayscale it should be noted that many of the images presented in the figures are color images. For example, in ASE and ASSE the intensity of the strain and its orientation are described using a color scale that mimics the visible light spectrum. Positive values appear on the top half of the scale and are represented by colors that decrease in intensity from red to green. Negative values appear at the bottom half of the scale and are represented by colors that decrease intensity from green to blue.

For all examples, the in vivo human tissue data were previously acquired at the University of Vermont by Dr. Brian Garra (under a different protocol). Altogether, a total of 134 embodiments consisted of 35 malignant tumors, 33 benign fibroadenomas and 66 other benign conditions. All the embodiments were pathologically confirmed. The data were acquired using an HDI-1000 (ATL-Philips Inc, Bothell, Wash.) scanner. The acquisition protocol used involved multi-compression, with a step size of 0.25%. The total compression was 5% and thus each human tissue ASSE shown in this disclosure is a multi-compression average of 20 realizations. These ASSEs were generated from the same algorithms used in processing the data obtained in the studies involving the use of tissue phantoms (see Example 2).

### Example 1

Modeling of benign and malignant breast lesions: In order to model realistic shapes that represent benign and malignant breast lesions sonographic shapes from an existing database of in vivo data acquired at an earlier time point were used. Example cases of pathologically-confirmed fibroadenoma and of a cancer where the lesions were asymmetric were identified and subject to analysis using a disclosed methods.

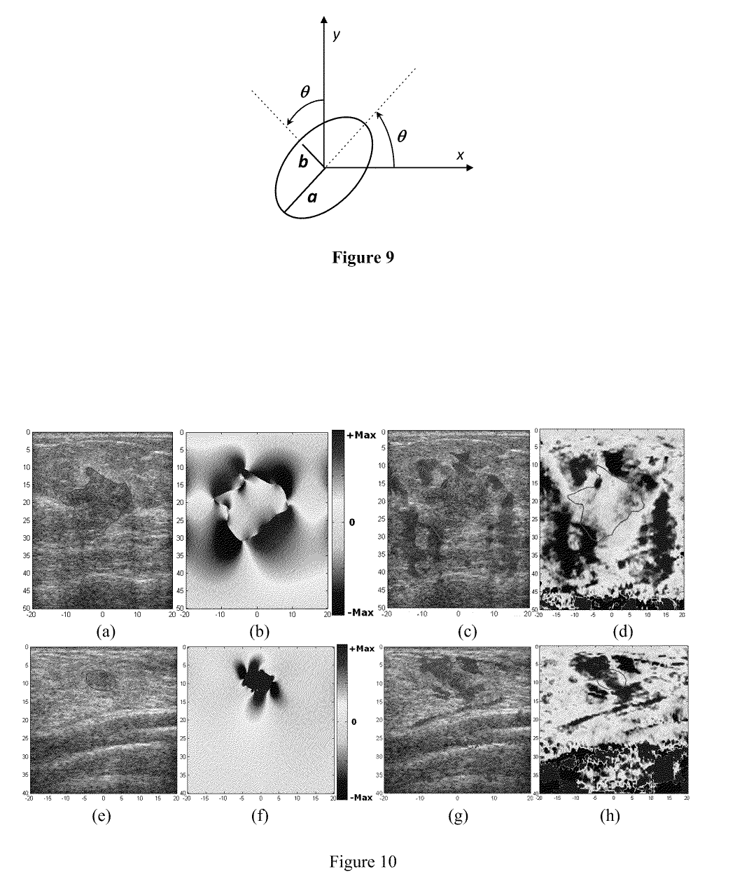

A 2D plane-strain model was built by using the Finite Element Modeling (FEM) software ANSYS® (Ansys Inc, Canonsburg, Pa.). The lesion's outline was manually traced by one of the authors (BG) on the sonogram and transferred to the finite element software using IGES file generation (Initial Graphics Exchange Specification: Claxton 2007, Matlab file:igesout.m. IGESOUT converts nurbs surfaces, curves and polylines to a single compact IGES format file, http://www.mathworks.com/matlabcentral/fileexchange/14470). To demonstrate the lesion geometry modeling method, an example of a pathologically-confirmed malignant breast lesion is shown in FIG. 9. The sonogram with the outlined lesion and the corresponding FEM mesh with the imported geometry are shown in FIG. 10. The imported geometry was embedded as a single stiff inclusion in a homogeneous, softer background with overall grid dimensions corresponding to the in vivo data size. The material properties of the inclusion and background were set to 42 Kpa and 21 Kpa, respectively. Thus, the inclusion was twice as stiff as the background material. A constant Poisson's ratio value of 0.495 was set for the inclusion and the background materials to model an essentially incompressible condition.

When an inclusion was embedded in a background, the inclusion/background interface was usually modeled by default as being firmly bonded in FEM software. Therefore, contact elements were introduced at the interface in order to be able to change the degree of bonding at the interface. One attribute of the contact elements is the coefficient of friction, a dimensionless constant usually ranging between 0 and 1, which can be changed to simulate different degrees of bonding at the interface. The lower limit on the coefficient of friction for a loosely bonded inclusion is zero, while a firmly bonded inclusion is considered to have infinite friction coefficient. For the FEM based on fibroadenoma-derived geometry, the inclusion boundary was modeled as loosely-bonded by assigning a very low coefficient of friction (μ) value of 0.01 to the contact element. The cancer-derived inclusion boundary was modeled as being firmly-bonded (μ=∞).

Inclusion geometry and orientation: The inclusion geometry in FEM based on in vivo fibroadenoma and cancer lesion provided an example case of asymmetric shape. In order to systematically investigate the ASSE at other inclusion asymmetries additional FE models were constructed by simplifying the asymmetry of cancer due to its irregular shape to that of asymmetry due to non-normally oriented ellipse because it was impractical to vary the asymmetry of the irregular shape in a consistent manner. However, cancers were modeled as firmly-bonded inclusions. The geometry consisted of a single stiff elliptical inclusion embedded in a square homogeneous softer background. The materials' properties of the inclusion and the background differed only in terms of their Young's moduli, and both had the same Poisson's ratio value of 0.495. The dimensions of the square background region were set at 40 mm by 40 mm.

Elliptical asymmetry was studied in terms of aspect the ratio (a/b) and the angle of orientation (θ) with respect to the axis of compression (y) as shown in FIG. 9. The aspect ratio allowed the characterization of the sensitivity of the axial shear strain fill-in to inclusion shape changes from symmetric (a/b=1) to asymmetric (a/b≠1). The angle in the model allowed us to create asymmetry for an elliptical inclusion (a/b≠1). Firmly-bonded and loosely-bonded inclusions at several different values of (a/b) and θ were generated. The effect of θ was studied for an inclusion model with aspect ratio (a/b)=1.5. The effect of aspect ratio was studied for the inclusion at orientation (θ)=45°. The inclusion-background modulus contrast for all the cases was fixed at 2:1, modeling a stiff inclusion.

Elastographic compression and image generation: The FE models were compressed from the top to apply a uniform axial strain. The node at the axis of lateral symmetry at the bottom of the model and the node at the center of the inclusion were confined in the lateral direction to avoid any rigid motion of the whole model. The pre- and post-compression node coordinate positions were saved and processed in MATLAB® (Mathworks, MA) to compute the axial-shear strain image using Eqn. 1 given below.

$${ɛ}_{axial - shear} = ( \frac{\partial v}{\partial x} ) \qquad ( 1 )$$

Where, v is the displacement along direction of compression (axial) and x is the lateral direction.

For the in vivo examples, pre- and post-compression RF frames were processed using a proprietary multi level coarse-to-fine 2D block matching algorithm implemented in Matlab® (Mathworks, MA) to compute the displacement map. The ASSEs were generated by extending the staggered-strain estimation method (see for example, Thitaikumar A, Mobbs L M, Kraemer-Chant C M, Garra B S and Ophir J. Breast tumor classification using axial shear strain elastography: a feasibility study. Phys Med Biol 2008; 53: 4809-4823). A 5×5 median filtering was applied to the displacement maps before estimating the ASSE.

ASSE Analysis: The influence of the asymmetry parameter (θ) and the aspect ratio (a/b) on the axial-shear strain fill-in inside the inclusion was studied systematically. A metric, percent fill-in (% fill-in), was defined which was calculated as the percentage of the inclusion's interior area that experienced axial-shear strain. The formula is given below in Eqn. 2 and where, axial-shear strain threshold stipulates the value of the pixel above which the pixel is considered ‘non-zero’. The axial-shear strain segmentation threshold used in Eqn. 1 was defined as 25% of the applied axial strain. This segmentation threshold was chosen to be consistent with that which had been determined for circular inclusions.

$$\% fill \text{-} in = ( \frac{\begin{matrix}number of pixels greater \\ than Axial shear strain threshold\end{matrix}}{total number of pixels inside the inclusion} ) \times 100 \qquad ( 2 )$$

US simulations: In order to determine that the features observed in the ideal mechanical FEM prediction are realized when using a band limited ultrasound pulse and a finite US beam in the presence of additive noise, the following US simulation study was carried out using MATLAB®. A shift-invariant Gaussian-modulated cosine pulse in the axial direction and Gaussian beam profile in the lateral direction were used to simulate the 2-D transducer point spread function (PSF).

The following simulation parameters were chosen to closely correspond to those of the SONIX-500 scanner (Ultrasonix Inc, Richmond, BC), which was later used for confirmatory phantom studies. The center frequency was set at 10 MHz with an RF sampling rate of 40 MHz while the fractional bandwidth was kept fixed at 50% of the center frequency. The −3 dB beamwidth of the Gaussian beam profile in the lateral direction was set to 1 mm. The simulations were performed assuming a speed of sound of 1540 ms⁻¹. The pre-compression RF signal was simulated by convolving the 2-D PSF with the scattering function. The scattering function was modeled by a normal distribution of a sufficient number of point scatterer amplitudes (as described in, for example, Meunier J and Bertrand M. Echographic image mean gray level changes with tissue dynamics: A system-based model study. IEEE Trans Biomed Eng 1995; 42 (4): 403-410 and Meunier J and Bertrand M. Ultrasonic texture motion analysis: Theory and simulation. IEEE Trans Ultrasonics Ferroelec Freq Control 1995; 14 (2): 293-300) to result in fully-developed speckle. The nodal displacement profile calculated from the FEA software was used to define the post-compression position of the point scatterers. The displaced scatterers were convolved with the original PSF to simulate the post-compression RF signal. In addition, an appropriate amount of uncorrelated random white noise was added to set the sonographic signal-to-noise ratio (SNRₛ) to 40 dB within the pass-band of the transducer.

The simulated pre-compression and the corresponding temporally-stretched post-compression RF signals were segmented and used in an adaptive strain estimation algorithm with lateral displacement correction to estimate the axial displacement. The local axial-shear strains were estimated as the gradients of the axial displacements in the lateral direction. A cross-correlation window length of 1 mm and a shift of 50% of the window length were used unless otherwise stated. A total of 15 independent realizations of the axial-shear elastograms were simulated for each condition by changing the scatterer distribution randomly. The calculated means and standard deviations in the subsequent plots correspond to 15 realizations.

Inclusion orientations between 0° and 90° were considered for the studies. The general behavior of the axial-shear strain pattern for orientations between 90° and 180° remains the same as between 0° and 90°, but the direction of axial-shear strain will be reversed. For example, positive valued shear strains (which appear in red) in an ASSE due to an inclusion orientation of 135° will become negative valued shear strains (which appear in green) for an orientation of 45°.

Correspondence between FEM images and in vivo ASSEs: FIG. 10 illustrates a comparison of the axial-shear strain image predicted by the FEM for the inclusion geometries derived from the sonogram of the in vivo case, and actual in vivo ASSE. Observe that the FEM predicts the fill-in of axial-shear strain inside the loosely-bonded asymmetric-inclusion, as observed in the ASSE of the elliptical-shaped fibroadenoma case. Observe the presence of a highly-contrasting axial-shear strain zone along the lesion boundary (see FIGS. 10 g and h) that is nearly inconspicuous in the ideal FEM images (see FIG. 10f). This is due to the finite resolution limits of the US beam that broadens the infinitely small discontinuity at inclusion-background boundary of loosely-bonded FEM. This effect was also noted in US simulation work of loosely-bonded circular inclusions. FEM predicts no such fill-in of axial-shear strain inside a firmly-bonded asymmetric-inclusion, resembling the ASSE of the irregular-shaped cancer case. Also, note observations in a noisy region near the bottom of the image in FIG. 10h. This is due to low the signal-to-noise ratio of the RF data in that region.

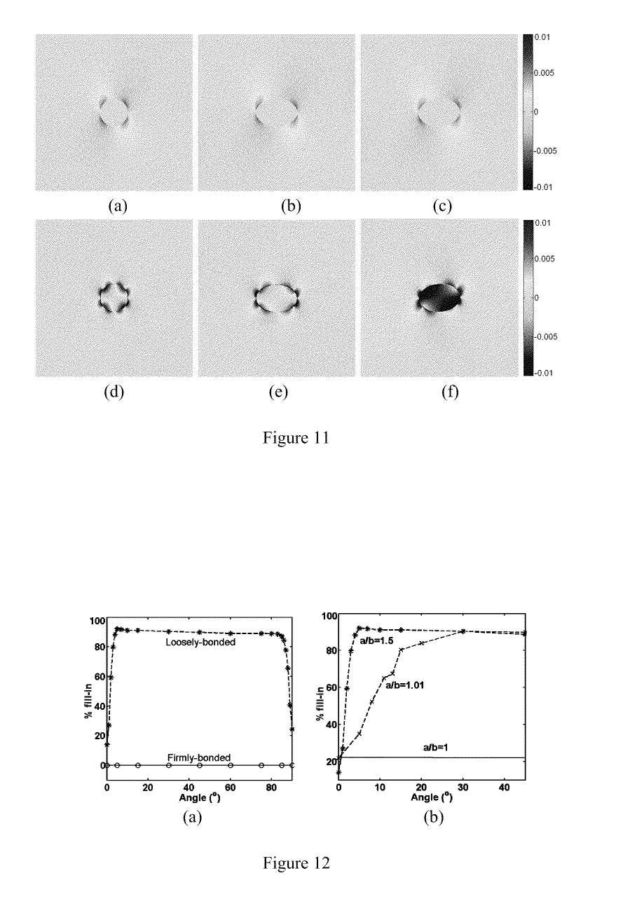

ASSEs due to circular and elliptical inclusions: The ASSEs due to symmetric (circular and normally-oriented ellipse) and asymmetric elliptical (non-normally oriented) inclusions that are either firmly-bonded or loosely-bonded to the background are compared in FIG. 11. The aspect ratio (a/b) and orientation (θ) of the elliptical inclusion were arbitrarily chosen for demonstration purposes.

Importance of inclusion orientation: A plot of percent fill-in as a function of the inclusion orientation (θ), for the two different bonding conditions are shown in FIG. 12. In the loosely-bonded case, axial-shear strain fills-in the interior of the inclusion rapidly. Beyond a ˜5° orientation, more than 80% of the inclusion is already filled with axial-shear strain that remains nearly constant until the inclusion becomes almost parallel to the compression axis (i.e. θ=90°). Note that this fill-in reaches a maximum of only about 90% due to the segmentation method adopted, and is explained as follows. The loosely-bonded boundary condition is modeled to allow slip at the boundary using theoretical contact elements. This gives rise to displacement discontinuity at the loosely-bonded boundary, which in turn leads to highly-contrasting axial-shear strain areas along the boundary. This contrasting axial-shear strain area at the boundary was not considered to be fill-in, but these pixels were considered part of the inclusion. Thus, it reduced the percentage of axial-shear strain area from the inclusion area. On the same plot (FIG. 12a), it can be observed that for the firmly-bonded case the axial-shear strains were completely absent inside the inclusion. FIG. 12b represents a magnified form of the plot shown in FIG. 12a that was obtained by restricting the x-axis to a smaller range of angles and adding two different aspect ratios (a/b). Only the loosely-bonded inclusion case was considered here. This plot highlights the influence of the (a/b) value on the critical angle at which fill-in occurs (i.e., the minimum angle at which the axial-shear strain fill-in value attains a certain (e.g., 50%) maximum value).

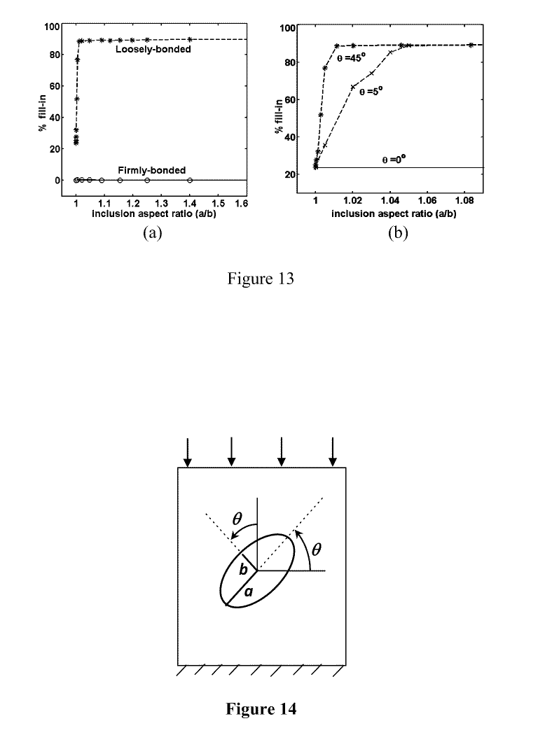

Importance of aspect ratio: The effects of the aspect ratio (a/b) on the percent fill-in are shown in FIG. 13a for the two different bonding conditions. The axial-shear strains fills in most of the interior of the inclusion only for a loosely-bonded inclusion. By contrast, it can be observed that the axial-shear strain distributions were absent inside the elliptical inclusion for a firmly-bonded inclusion at any aspect ratio. FIG. 13b illustrates a magnified form of the plot shown in FIG. 13a obtained by restricting the x-axis (range of a/b value) but, at two different inclusion orientations (θ). Here again, only the loosely-bonded inclusion case is considered. This plot highlights the influence of θ on the critical value of (a/b) at which the axial-shear strain fill-in the inclusion. This behavior is very sensitive to the change of the shape of the inclusion from circular to elliptical. In fact, even with a small change away from circularity, the percent fill-in value is about 90% of inclusion area. Here again, the fill-in is not 100% for the reasons explained earlier.

### Example 2

In the present example, the use of the axial-shear strain fill-in effect to characterize inclusion boundaries is validated using tissue mimicking gelatin-phantoms and was extended to demonstrate the existence of the axial-shear strain fill-in effect in fibroadenomas as well as some other benign conditions and to demonstrate that the lace of the axial-shear strain fill-in effect is consistant with the presence of a malignant lesion.

Imaging phantoms (phantoms) are specially designed objects that used as a substitute to live or cadaver subjects in human medical imaging. Phantoms are used to evaluate an imaging device or a technique and should respond in a manner similar to that of human tissues and organs under the specific imaging modality.

Phantom Preparation: All the tissue-mimicking phantoms used for these studies were made of gelatin-agar-water mixtures. The agar particles in the mixture acted as the acoustic scatterers. The recipe for manufacturing these phantoms with realistic acoustic properties has been described by many research groups (see for example, Kallel F, Prihoda C D, Ophir J. Contrast-transfer efficiency for continuously varying tissue moduli: simulation and phantom validation. Ultras Med Biol 2001; 27:1115-1125). Phantom geometry consisted of a homogeneous cuboid with a cylindrical (elliptical cross-section) inclusion of contrasting Young's modulus running horizontally through the center of the cuboid. The long-axis and short-axis radii of the elliptical inclusion were 17 mm and 10 mm, respectively. A plastic mold of cubic shape with provision for inserting a thin-walled cylindrical die at the center was manufactured for this purpose. The width and length of the cuboid measured 80 mm×90 mm while the height could be adjusted. The background material was prepared by mixing 5% by weight gelatin and 3% by weight agar in de-ionized water at 80° C. while the cylindrical inclusion material was prepared by mixing 10% by weight gelatin and 3% by weight agar. The agar-gelatin-water mixture that makes up the background was poured into the cubic mould with the cylindrical die in place and allowed to cool at 4° C. for approximately 12 hours. Thereafter, the procedure varied slightly depending on whether a firmly-bonded or loosely-bonded inclusion was manufactured. To obtain a firmly-bonded inclusion, the cylindrical die was carefully removed from the background and the agar-gelatin-water mixture that makes up the inclusion was poured into the cylindrical hole in the background and allowed to cool further at 4° C. for approximately 12 hours. To obtain a loosely-bonded inclusion, the hollow cylindrical die was carefully removed from the background and the agar-gelatin-water mixture that makes up the inclusion was poured into the die and cooled at 4° C. for approximately 12 hours to form a separate inclusion. After the formed inclusion was removed from the die, it was inserted carefully into the background, such that a loosely-bonded boundary was formed by the outer surface of the cylindrical inclusion and the inner surface of the matching hole in the background. The preferred phantom contained inclusions that are stiffer than the background. No independent Young's modulus measurements were made on phantoms. However, based on the relative gelatin concentrations in the background and the inclusion, the expected inclusion-background modulus ratio was ˜2.

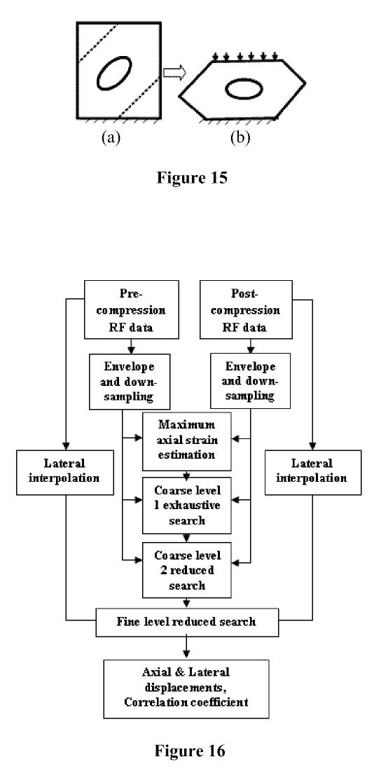

Four different phantoms were constructed for this study. Each phantom had either a firmly- or loosely-bonded inclusion. The inclusions in all the phantoms had a cross-sectional aspect ratio (a/b) ˜0.7. A sketch of the phantom cross-sectional geometry is given in FIG. 14. The dimensions and inclusion-orientation in the four different phantoms were for the firmly-bonded inclusion: phantom height=65 mm, width=80 mm, θ=0°; for the other firmly-bonded inclusion: phantom height=60 mm, width=80 mm, θ˜45°; for first the loosely-bonded inclusion: phantom height=65 mm, width=80 mm, θ=0°; and for the second loosely-bonded inclusion: phantom height=80 mm, width=80 mm, θ˜′45°. A 5^{th} phantom by created by cutting phantom 4 after it was scanned, as shown in FIG. 15, such that the inclusion was now oriented at θ=0°. This was done to allow a change in the orientation angle (θ) between of axis of compression and inclusion in the same phantom. Note the height of the phantom was reduced from 80 mm to 60 mm as a result.

A Sonix 500-RP US scanner, with a 128-element linear array, 10 MHz center frequency was used for data acquisition. The RF sampling rate was 40 MHz. The transducer was attached to a compressor plate (80 mm×40 mm), which was in turn attached to a precision computer-driven motion controller. Phantoms were submerged in water during imaging. The phantoms were compressed from the top by a small amount that corresponded to an applied strain of 1%. Pre- and post-compression RF data sets were acquired from each phantom at 10 different planes along the elevational direction. The planes were separated by at least 3 mm (˜elevational beam width) to obtain independent frames for subsequent averaging.

Displacement tracking and Axial Shear Strain Elastogram (ASSE) generation. The displacement tracking algorithm consisted of multilevel, coarse to fine, 2D block matching scheme. The principal difference was the use of sum square difference (SSD) similarity measure in the coarse level on the envelope of the signal, followed by the cross-correlation (CC) measure on the RF signal in the fine level around the SSD-tracked coarse displacement region. A flow chart of the algorithm is given in FIG. 16.

After obtaining the axial displacement using the 2D tracking method explained above, the ASSE was generated by extending the staggered strain estimation (Srinivasan et al. 2002, ibid) for axial strain estimation. In the extended version, the axial displacements are staggered along the lateral direction to estimate the axial-shear strain (ThitaiKumar et al. 2007, ibid).

In vivo examples. Data from an existing database of in vivo breast fibroadenomas and cancers were identified that had approximate elliptical shape and a non-normal orientation with respect to axis of compression. The database was acquired during an earlier study without having any information on ASSE where the quasi-static compression was given in one direction (axial). Therefore, only those cases that happen to be at a non-normal orientation had potential to be part of the current study to demonstrate the existence of the axial-shear strain fill-in phenomenon in benign fibroadenomas as described earlier. The data acquisition details and processing algorithms are described in the following paragraph.

The data were previously acquired at the University of Vermont by Dr. Brian Garra for a different project. Altogether, a total of 134 cases consisted of 35 malignant tumors, 33 benign fibroadenomas and 66 other benign conditions. All the cases were pathologically confirmed. The data were acquired using an HDI-1000 (ATL-Philips Inc, Bothell, Wash.) scanner. The acquisition protocol involved multi-compression, with a step size of 0.25%. The total compression was 5% and thus each ASSE shown in this disclosure is a multi-compression average of 20 realizations. The ASSEs were generated from the same algorithms used for experimental phantom data processing as described earlier.

The axial-shear strain pattern for orientations between 90° and 180° was the same as between 0° and 90°, but the direction of the axial-shear strain was reversed. For example, positive valued regions in ASSE for an inclusion orientation of 135° will become negative valued for an orientation of 45° (135°−90°).

The finite resolution of the US system is determined by its finite point-spread function (PSF) resulted in the appearance of a high-contrasting finite axial-shear strain zone along the loosely-bonded inclusion-background boundary, which was infinitesimally small. This thin, high-contrast axial-shear strain zone was of the opposite polarity of that of the axial-shear strain values inside the inclusion.

Analysis using Phantoms. Among the various elastographic images, only the ASSE is displayed along with the corresponding sonogram. Further, all the phantom images (sonogram and ASSE) were truncated to correspond to 60 mm depth for easy visual dimensional comparison.

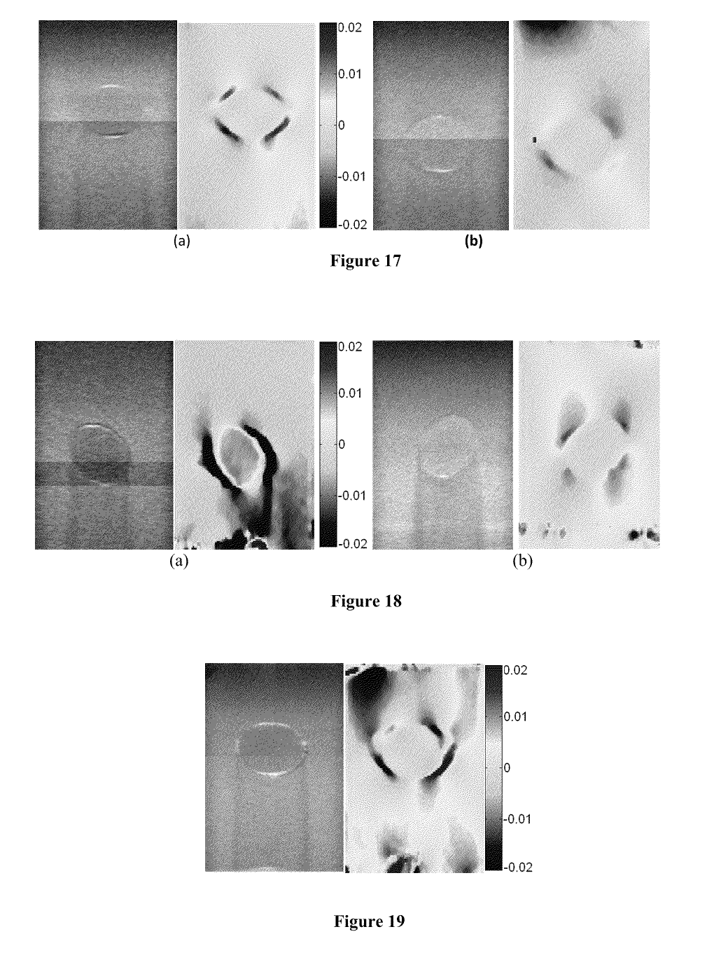

The ASSE from phantoms 1 and 3, where the inclusion is not inclined (i.e., θ=0°), are shown in FIG. 17. It can be observed that axial-shear strain does not fill-in the inclusion in both the firmly-bonded and loosely-bonded elliptical inclusion phantoms.

FIG. 18 shows the ASSE from phantoms 2 and 4, where the inclusion is oriented at an angle (θ˜=+/−45°). Note that axial-shear strain fills-in the inclusion only for the loosely-bonded elliptical inclusion phantom (FIG. 17a) and not the firmly-bonded elliptical inclusion phantom (FIG. 17b).

The ASSE from the 5^{th} phantom is shown in FIG. 19 This phantom was altered from the original phantom 4 such that the inclusion is now oriented at θ=0°. In other words, this is the same as phantom 4 (θ˜−45°) but, now there is a change in direction of compression so that the inclusion has no relative orientation (i.e., θ=0°). It can be seen that the axial-shear strain that filled-in the inclusion in phantom 4 (FIG. 18a) is now absent in phantom 5. The axial-shear strain distribution for phantom 5 resembles the ASSE of phantom 3 shown in FIG. 17. It is worth noting that in this special case when the elliptical-inclusion is not oriented with respect to direction of compression, it becomes symmetric and the axial-shear strain distribution pattern behaves in a fashion similar to that reported for circular-shaped inclusions.

In vivo examples: Several fibroadenomas having approximately elliptical shapes and an orientation non-normal to axis of compression were identified. As were several cases of other benign lesions and cancers that happen to approximate the shape and orientation criteria. A few representative findings are described for each type of lesion.

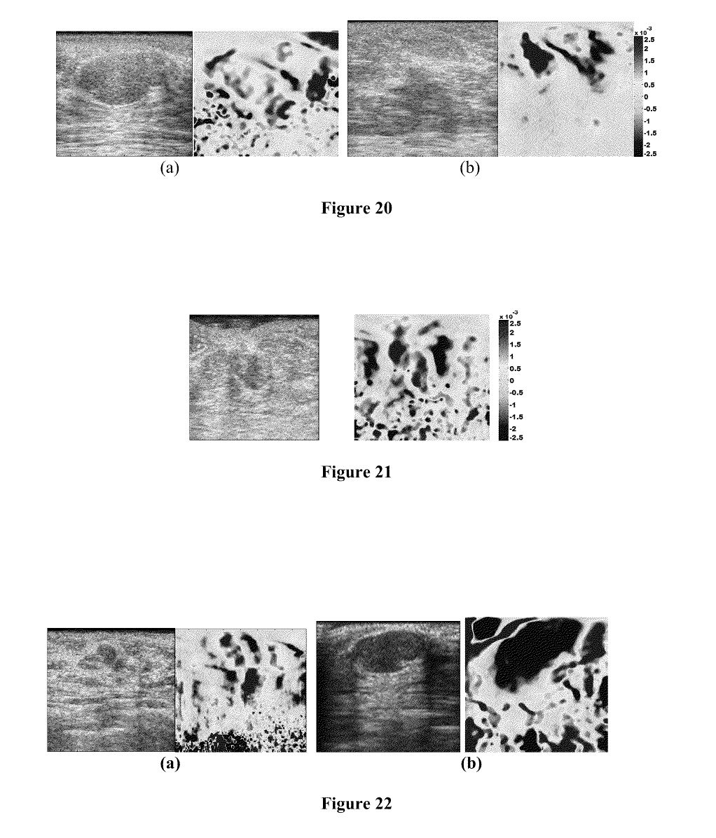

FIG. 20 compares and contrasts the ASSE from one fibroadenoma and one cancer case. The fill-in of the axial-shear strain was clearly noted in fibroadenoma (FIG. 20a) but not in cancer (FIG. 20b). It can be seen that these ASSEs resemble those from corresponding phantom models shown earlier in FIGS. 18a and b.

FIG. 21 is an additional example of ASSE of fibroadenoma along with a corresponding sonogram. It can be observed that the ASSE of this fibroadenoma follows the axial-shear strain distribution pattern of the loosely-bonded elliptical inclusion phantoms shown in FIG. 18a.

FIG. 22 shows example ASSE of benign lesions that are not fibroadenomas but which satisfied the shape and orientation requirement. It can be seen that even for these cases, the axial-shear strain distribution pattern resemble that of loosely-bonded elliptical inclusion. Indicating that these benign lesions were all loosely-bonded to the surrounding tissue as would be expected given the nature of benign lesions which lack the invasive nature of malignant lesions and may even be encapsulated.

In this section it was shown that axial-shear strain due to firmly-bonded elliptical inclusions occurred only outside the inclusion. During the analysis of phantom ellipses axial-shear strains filled-in the interior in loosely-bonded elliptical inclusions exclusively when obtained at non-normal orientations. Thus one may or may not observe a fill-in inside a loosely-bonded elliptical inclusion depending on its relative orientation (θ) relative to the axis of compression but by changing the axis of compression, as in the case of remanufacturing phantom 5 from phantom 4 or by varying the inclusion orientation by changing the axis of compression. With the in vivo cases, axial-shear strain fill-in was observed inside the fibroadenoma, while no such fill-in was observed in the case of malignant tumor. Similar findings, the fill-in of axial-shear strain, were observed with benign cases that were not fibroadenomas, thus the technique can be generally applied.

Notice that the axial-shear strain fill-in in the ASSE of some of the benign fibroadenomas was not as complete or as highly intense as it was in experimental phantom cases. This can be attributed to the fact that the relative orientation was not large enough and the extent of fill-in of axial-shear strain depends on both aspect ratio (a/b) and relative orientation of the elliptical inclusions used. The less elliptical the inclusion, the larger the relative orientation required before complete fill-in can be observed. Therefore, it is likely that these benign fibroadenoma cases did not have a high enough relative orientation for the given aspect ratio to have a complete fill-in of axial-shear strain inside the lesion.

Currently the Breast Imaging-Reporting and Data System (BI-RADS, ACR 2003) uses several sonographic features based on qualitative descriptions of the shape, contour, margin, and ultrasonic echogenicity of the lesions. The inter-observer variability due to these qualitative descriptors has been a concerning issue in the characterization of solid breast masses on sonography. Because axial-shear strain fill-in inside an elliptical inclusion is unique to those that are loosely-bonded, it may therefore also be a unique signature of benign conditions. Thus, it may be a sign of benignity of benign breast lesions (e.g. fibroadenomas) that are known to be stiff, elongated and loosely-bonded to the host tissues. Thus the use of the presently disclosed methods and presence or absence of axial-shear strain fill-in inside a lesion should reduce the qualitative inter-observer variability in the BI-RADS paradigm.

### Example 3

HIFU has become a promising noninvasive technique to thermally ablate and destroy volume of tissue lying deep under the skin surface. HIFU technique is synonymously used for focused ultrasound surgery (FUS), which was first investigated and described for neurosurgical applications about five decades ago. In the last few decades, the HIFU technique has found several applications that include, among others, ablation of tumors in prostate and other organs such as, but not limited to, liver, kidney, bladder, and breast as well as for arresting hemorrhage.

The success of the HIFU procedure requires that the progress of the treatment be accurately visualized. There is a longstanding desire to have a unified system for HIFU treatment and inexpensive real-time lesion visualization utilizing ultrasound based methodologies. However, the extent of the HIFU lesion is difficult to quantify with current B-mode imaging (sonography) techniques because of lack of contrast between HIFU lesions and normal tissue boundaries, as well as shadows that limit the entire view of the lesion (as seen for example in FIG. 22(a)).

The technique involves acquiring US (RF/envelope) signals from an imaging plane before and after the application of a small (˜1%) quasi-static compression. Typically, the pre- and post-compression frames are processed to generate images of local strain, commonly known as elastograms. When the elastogram depicts axial strain values, it is referred to as an axial strain elastogram (ASE) (see, for example, U.S. Pat. No. 7,779,692). The feasibility of using ASE to detect HIFU lesion has been demonstrated in ex vivo animal model as well as ex vivo human prostate. Because the associated protein denaturation causes the HIFU lesion to be stiffer than the surrounding soft tissue it was a challenge to define the exact boundaries of the HIFU-lesion using only ASE.

ASSE was used to visualize the boundaries of breast lesions (see the prior Examples). In this technique, the axial-shear strain experienced by the tissue element due to quasi-static compression (as in elastography) is imaged and referred to as ASSE. Axial-shear strains are generated at the boundaries of a firmly-bonded inclusion, and thus producing contrast to visualize the boundary directly. In some embodiments of the present disclosure, ASSE is used to reliably visualize and identify HIFU-lesion boundaries.

Recently, axial-shear strain elastography has been introduced as a method to visualize the boundaries of spherical inclusions (Thitaikumar A, Krouskop T A, Garra B S, and Ophir J. Visualization of bonding at an inclusion boundary using axial-shear strain elastography: A feasibility study. Phys Med Biol; 52: 2615-2633, 2007). In this technique, the axial-shear strain experienced by the tissue element due to quasi-static compression (as in elastography) was imaged and referred to as ASSE. As exemplified above axial-shear strains are generated at the boundaries of a firmly-bonded inclusion, and produce a contrast that allows one to visualize the boundary directly. The following example is provided to demonstrate that a derived method can be used to establish reliable HIFU lesion boundary visualization and differentiate treated from untreated tissue.

Original data acquisition and data reprocessing. The data for this example were acquired under a previous protocol (as described in Righetti, et al. 1999, ibid). The samples consisted of excised canine livers with thermal lesions produced by a prototype MR-compatible HIFU system (GE Medical System, Milwaukee, Wis.). After thermal exposure the samples were cast in a gelatin block and placed in the compression apparatus. The elastographic experiments were conducted using a linear array scanner (Diasonics Spectra II, Santa Clara, Calif.) operating at a 5 MHz center frequency. The samples were scanned using a multi-compression scheme with a step size of 0.5%. For each step the RF signals were digitized using an 8-bit digitizer (Lecroy Corp., Chestnut Ridge, N.Y.) operating at a 48 MHz sampling frequency.

Validation of the use of ASSE to characterize HIFU lesions: For the present analysis, the original pre- and post-compression RF data were reprocessed using a multilevel coarse-to-fine 2D block-matching algorithm (As described in the prior Examples) to compute the displacement map. ASE and ASSE were generated from the displacement estimates using a least squares strain estimator (LSQE) with a kernel size of ˜4 mm and staggered-strain estimation, respectively.

Characterization of lesion area: The data archive consisted of samples containing a single HIFU lesion and multiple HIFU lesions. For each of the single lesion samples, the lesion boundary was determined independently from the ASE and ASSE at various isointensity contour thresholds, and the area of the enclosed lesion was computed.

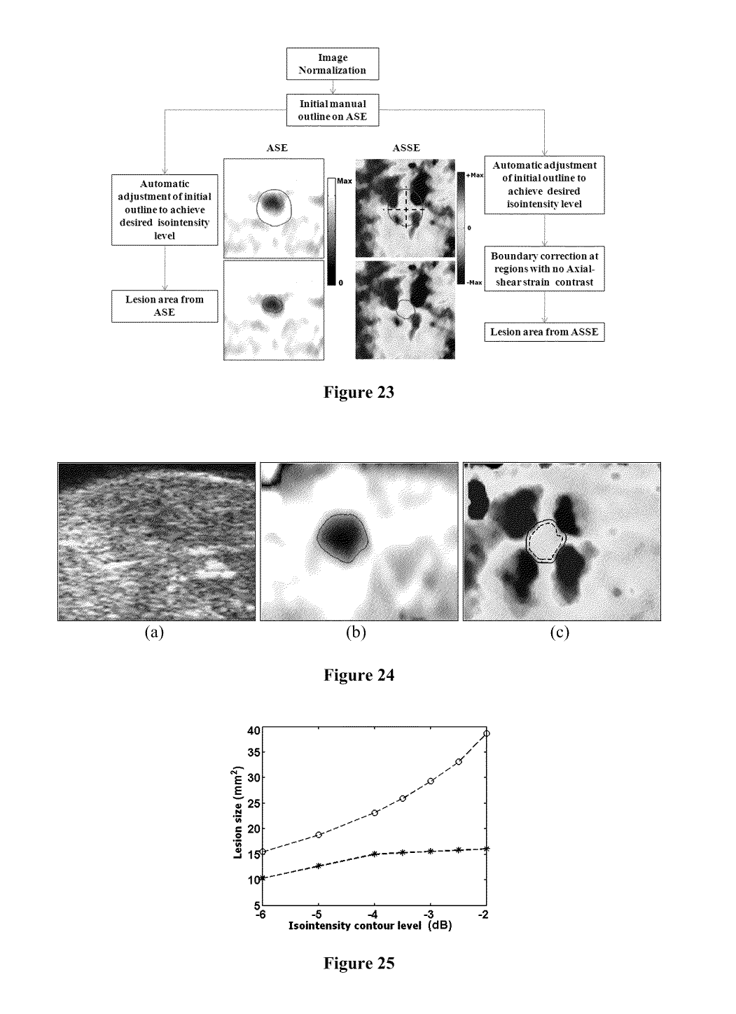

In ASE the level of axial strain inside the lesion and in the surrounding background are contrasted to produce the isointensity contour level of the strain contrast (between the lesion and the surrounding) which defines the lesion boundary for that level. The level of axial strain was varied from −2 dB to −6 dB. ASSE is based on a strain contrast exists between the finite axial-shear strain regions at the lesion boundary and lack of axial-shear strain inside the lesion. The isointensity contour level of the axial-shear strain contrast defines the lesion boundary for that level. The lesion boundary on ASE and ASSE at an isointensity contour level was determined in the following manner, which is also diagramed in FIG. 23.

First, both images were normalized based on the high-valued pixels that were of good quality (i.e., having corresponding correlation coefficient value >0.75) in the respective images. For the ASSEs the positive and negative valued axial-shear strain regions were normalized separately. This normalization procedure is necessary to obtain consistent depiction of the lesion at a particular strain isointensity threshold value. Second, the lesion on the ASE was manually outlined for each case and used as a starting boundary position. This initial outline was automatically adjusted (reduced or increased asymmetrically) to achieve the desired isointensity contour level. Although the lesion itself is visible in ASE, it is not visible in the ASSE and there exist regions where there is no axial-shear strain contrast between the host tissue and the lesion (usually in regions close to 0°, 90°, 180°, and 360°). In order to obtain a closed lesion boundary the initial outline from the ASE was used as a staring boundary position on ASSE. This initial outline was automatically adjusted (reduced) to achieve the desired isointensity contour level. Finally, the area of the lesion from ASE and ASSE were obtained at various isointensity contour levels.

For samples containing multiple lesions, the resulting ASSE was visually analyzed to identify axial-shear strain zones of interest. In order to validate and characterize the system used to obtain the axial-shear strain zones observed in the ASSE from ex-vivo tissue samples, Finite Element Models (FEM) with two stiff-inclusions (both were twice as stiff as the background) were constructed. The FEM were done using ANSYS® (Ansys Inc, Canonsburg, Pa.) assuming a 2D plane strain model. Four models were built, each having a different relative position for the two inclusions. In all the models the inclusions were 10 mm in diameter and the overall geometry was 40 mm×40 mm. In the first model, the inclusions were located at the same depth but were separated laterally by 10 mm. The two inclusions in the second model were at the same depth but separated by only 0.1 mm, and in the third model the two inclusions overlapped 50% laterally. In the fourth model, the inclusions were not only separated by 0.1 mm laterally but were also located at different depths (2.5 mm offset between their centers). The models were subjected to 1% compression from the top. The pre- and post-compression node positions were imported into Matlab® (Mathworks, MA) and the axial-shear strains were computed as the gradient of the axial-displacement in lateral direction.

Visualization of a Single HIFU-lesion: A total of five single lesion cases were characterized. The HIFU-induced lesion area was estimated from ASE and ASSE separately, based on the lesion boundary determined by the isointensity contour threshold values ranging from −2 dB to −6 dB. An illustrative flow diagram that demonstrates the iso-intensity contour segmentation method used in the HIFU-induced lesion area estimation from the ASE (left column, gray scale) and ASSE (right column, color scale: FIG. 23) The automatic lesion boundary determined at an isointensity threshold value of −3 dB is shown in the on bottom row.

A Sonogram, an ASE and an ASSE of an exemplary case of a HIFU-lesion are shown in FIG. 24. The lesion boundary corresponding to two isointensity contours of −2 dB (solid line) and −6 dB (dashed line) are shown on the elastograms. When the ASE and the ASSE are compared the difference between the enclosed areas at the two contour levels can be readily appreciated. The corresponding sonogram in FIG. 24a suggests an indistinct hypoechoic area in which it is difficult to clearly define a HIFU lesion. Therefore no estimated lesion area could be determined in the sonagram. However, estimated HIFU-induced lesion areas, identified throughout a range of isointensity contour levels, were determined for both with ASE (circles) and ASSE (asterisks) were plotted (FIG. 25). From this plot, it is clear that the HIFU-lesion area estimation and boundary visualization using ASE is more variable over the image display dynamic range than that obtained using ASSE. Table 1 compares the mean and standard deviation values of the estimated lesion area obtained at seven isointensity contour values between −2 dB to −6 dB, from ASE and ASSE for the 5 single-lesion cases analyzed. With ASE, the overall variation in estimated HIFU-lesion area fell between approximately 31 to 49% while the corresponding variation for the lesion area estimated with ASSE were much more consistent, with variations ranging between approximately 15 to 20%. This indicates that the estimation of the HIFU-induced lesion area (or lesion boundary visualization) using ASSE is less sensitive to isointensity threshold selection and that the ASSE method is more robust than the ASE-based method for lesion boundary visualization and lesion area estimation.

TABLE 1

| | |
|---|---|
| Mean (±std) values (mm²) | Variation (%) |

| Case # | ASE | ASSE | ASE | ASSE |
|---|---|---|---|---|
| 1 | 13.6 (±6.6) | 10.7 (±2.2) | 48.5 | 20.6 |
| 2 | 42.3 (±14.2) | 31.8 (±6.1) | 33.6 | 19.1 |
| 3 | 32.7 (±11.1) | 20.2 (±3.4) | 34.0 | 16.8 |
| 4 | 26.4 (±8.1) | 14.4 (±2.1) | 30.7 | 14.6 |
| 5 | 12.2 (±5.4) | 18.3 (±2.7) | 44.3 | 14.8 |

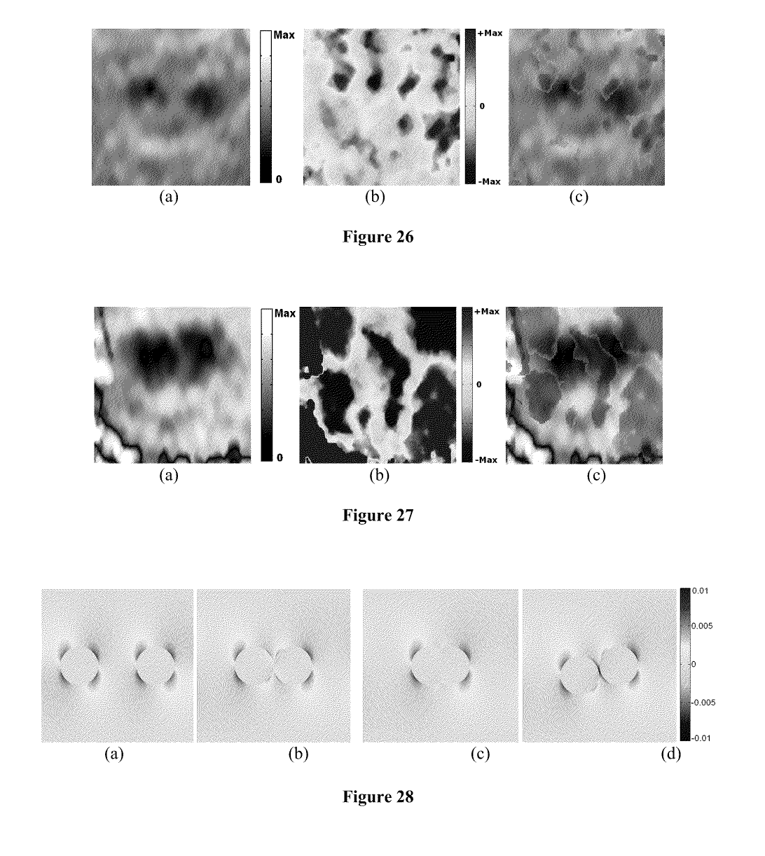

Analysis of Multiple (Two) HIFU-Lesions ASSEs of ex-vivo tissue samples: ASE and ASSE of a sample with two HIFU-lesions that are well separated are shown in FIG. 26. The two lesions are well identified individually on ASE as regions with low axial strain values shown in FIG. 26a. The characteristic positive (represented by colors from green to red signal) and negative (represented by colors from green to blue) axial-shear strain pattern at the lesion boundary clearly identifies the two lesions separately in the ASSE shown in FIG. 26b. In order to better visualize axial-shear strain distribution pattern in the context of the lesion visible on ASE, a composite image with color-overlay of ASSE on top of ASE is provided in FIG. 26c. Only those pixels from ASSE that were of good quality (corresponding correlation coefficients >0.75) and greater than a threshold value (50% of peak value) were color-overlaid in FIG. 26c.

When the two HIFU-lesions are close together, one obtains the images in ASE, shown in FIG. 27a, and ASSE, shown in FIG. 27b. In ASSE, unexpectedly, the negative axial-shear strain region of one lesion cancels the positive axial-shear strain region of the other, thus only 4 regions (2 per lesion) of finite axial-shear strain at the lesion boundary were seen instead of the 8 regions that would have been expected had the two lesions behaved independently (e.g. compare FIG. 26b and FIG. 27b). In addition, notice that ASSE enables high-contrast visualization of a ‘thin’ untreated region between the two lesions. The thin soft untreated region is barely visible between the two stiff lesions on ASE and with the image smoothing parameters commonly employed to reduce noise and improve the image quality in ASE, this minimal contrast can deteriorate further.

ASSE of models: Axial-shear strain maps from FEM for the 4 different models are shown in FIG. 28. Observe that the FEM mimic the axial-shear strain region associated with each inclusion appearing as being distinct when the inclusions are well separated as in FIG. 28a. However, the opposite polarity axial-shear regions start to cancel at the boundaries that comes close together as the inclusions are brought closer as seen in FIG. 28b before eventually vanishing when the two inclusions merge into one as seen in FIG. 28c. When the inclusions are vertically-offset, the locations of the axial-shear strain regions of the two inclusions are also offset in such a way that the axial-shear strains with the same polarity reinforce each other as seen in FIG. 28d. This gives rise to a high-contrast axial-shear strain zone at the thin (0.1 mm) soft region between the stiff inclusions. The ASSE observed for the ex-vivo samples (FIGS. 26 and 27) are consistent with FEM predictions shown in FIG. 28.

Using the disclosed methods of ASSE, a change in the contrast level threshold affects the size of the axial-shear strain region at the boundary. Thus ASSE differs from ASE in that, ASSE images the boundary of the lesion and not the lesion directly, as does ASE.

However, the location where finite axial-shear strains first appear outside the lesion, which determines the lesion boundary, remains stable. Therefore, lesion boundary visualization using ASSE is more robust than that identified using ASE. When multiple lesions were well separated spatially, their appearance on the corresponding ASE was unambiguous. In the corresponding ASSE, the axial-shear strain zones at the boundaries of each of these lesions were clearly identified.

When the pair of lesions came close together, the lesion images start to merge into one with ASE. The appearance using ASSE is interesting in that the axial-shear strain zones associated with each of the lesion boundaries vanishes at the boundary regions that come close together. Interestingly, the axial-shear zone corresponding to the ‘thin’ region between the two lesions was visualized at high-contrast. This behavior of ASSE may prove important in some applications. For example, HIFU treatment of a large tumor is typically achieved by multiple small exposures that cover the entire volume. Therefore, it is important to visualize the multiple smaller lesions and ensure that there remains no untreated region located between them. Clearly, the ASSE methods of the present disclosure are useful in such situations to visualize the thin untreated regions. Once the thin untreated (soft) region is treated, it is likely the two separate lesions will merge into one larger lesion and the high-contrast axial-shear zone in-between the lesions will also vanish. Thus, the ASSE methods of the present disclosure can be used to direct and monitor HIFU treatment.

Some of the presented examples utilize in vitro and ex vivo human and veterinary tumors and tissue samples to demonstrate the utility of the present methods with tumors, those of skill in the art would readily understand that these results are representative of those that would be obtained in vivo, particularly because the in vivo functionality of ASSE has already been demonstrated. Furthermore, although human and canine tissue is specifically exemplified, those of skill in the art would readily realize that these methods are of use in, but not limited to, humans, primates, laboratory animals and companion animals. The can also be generically utilized as described herein and in ways. Furthermore, while the presently disclosed examples concentrate on the medical uses of the disclosed invention, it is readily apparent that these methods have utility whenever one wishes to scrutinize a deformable target body for homogeneity or to search for an asymmetrical inhomogeneity within any deformable target body. Such uses include, but are not limited to, quality control analysis following the production of a deformable target body for which homogeneity is desired.

While preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, and so forth). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, and the like.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the preferred embodiments of the present invention. The discussion of a reference in this disclosure is not an admission that it is prior art to the present invention, especially any reference that may have a publication date after the priority date of this application. The disclosures of all patents, patent applications, and publications cited herein are hereby incorporated by reference, to the extent they provide exemplary, procedural or other details supplementary to those set forth herein.

What is claimed is:

1. A method for identifying the bonding characteristics of an inclusion within the normal tissue of an individual, the method comprising:
a) producing ultrasound axial-shear strain elastography images of a target region in said individual including or suspected of including an inclusion;
b) identifying from said axial-shear strain elastography image the presence of non-zero axial-shear strain within an inclusion and its surrounding normal tissue in said target region; and
c) calculating the non-zero axial-shear strain that is in the inclusion as a percent fill-in value, wherein a calculated percent fill-in value of less than 50% is indicative of an inclusion firmly bonded to its surrounding tissue.

2. The method of claim 1, wherein a percent fill-in value greater than 50% is indicative of an inclusion loosely bonded to its surrounding tissue.

3. The method of claim 1, further comprising:
d) after systematically changing the angle of compression relative to the inclusion repeating steps a-c, and;
e) determining a maximum percent fill-in value for the inclusion.

4. The method of claim 3 wherein in (d), said systematically changing the angle of compression comprises translating the transducer relative to the inclusion.

5. The method of claim 3 wherein in (d), said systematically changing the angle of compression comprises manipulating the transducer through a minimum of three Yaw angles relative to the inclusion.

6. The method of claim 3 further comprising (f) charting percent fill-in and the angle of compression relative to the inclusion.

7. A method of diagnosing an individual having or suspected of having a tumor, comprising:
a) producing an ultrasound axial-shear strain elastography image of a target region in said individual, said region including or suspected of including a tumor;
b) identifying from said axial-shear strain elastography image the presence of non-zero axial-shear strain within an inclusion and its surrounding tissue in said target region;
c) calculating the non-zero axial-shear strain that is in the inclusion as a percent fill-in value;
d) systematically changing the angle of compression relative to the tumor and repeating steps a-c;
e) determining a maximum percent fill-in value for the tumor.

8. The method of claim 7, wherein a maximum percent fill-in value of less than 20% is suggestive of a malignant tumor.

9. The method of claim 7, wherein a maximum percent fill-in value between 20% to 50% is less suggestive of a malignant tumor compared to a percent fill-in value of less than 20%.

10. The method of claim 7, wherein a maximum percent fill-in value between 50% to 80% is suggestive of a benign tumor.

11. The method of claim 7, wherein a maximum percent fill-in value of greater 80% is more suggestive of a benign tumor compared to a percent fill-in value between 50% to 80%.

12. A method of accurately identifying the margins of a tumor within normal tissue, comprising:
a) producing an ultrasound axial-shear strain elastography image of a target region in said individual, said region including or suspected of including a tumor;
b) identifying from said axial-shear strain elastography image the presence of non-zero axial-shear strain within an inclusion and its surrounding tissue in said target region;
c) calculating the non-zero axial-shear strain that is in the inclusion, as a percent fill-in value;
d) systematically changing the angle of compression relative to the tumor and repeating steps a-c;
e) combining the resulting calculations to create an overlapping image that accurately identifies the boundaries of the tumor; and
f) displaying said image.

13. A method of directing tumor therapy comprising performing the method of claim 12 on said individual during tumor therapy, in real-time.

14. The method of claim 13 wherein said tumor therapy is HIFU.

15. A method to identify a region of untreated tumor tissue located between a boundary of two treated tumor tissue regions within a target region that comprises:
a) producing an ultrasound axial-shear strain elastography image of a target region in said individual, said region including or suspected of including a tumor region that contains at least a first HIFU lesion and a second HIFU lesion;
b) identifying in said axial-shear strain elastography image the presence of the first HIFU lesion and the second HIFU lesion by the presence of a characteristic axial-shear strain pattern located at the boundary of both the first HIFU lesion and a second HIFU lesion;
c) identifying in said axial-shear strain elastography image the untreated tumor tissue region that lies between the first HIFU lesion and a second HIFU lesion; and
d) repeatedly applying HIFU to the identified untreated tumor tissue region until the boundary of the first HIFU lesion and the second HIFU lesion are united and the boundary appears within a real-time axial shear strain elastography image as an axial shear strain pattern that is characteristic of a single HIFU lesion.

16. The method of claim 15, further comprising:
e) systematically moving between HIFU lesions within the tumor region; and
f) repeating steps a-e until the entire tumor region has been treated.

17. A method of directing tumor therapy by identifying regions of untreated tumor tissue in an individual, said method comprising performing the method of claim 15 on said individual during tumor therapy, in real-time.
